(19) United States
(12) Patent Application Publication
(10) Pub. No.: US 2003/0100748 A1
(43) Pub. Date: May 29, 2003

(54) Plant promoter derived from luminal binding protein gene and methods for its use

(75) Inventors: Santosh Misra, Victoria (CA); Benjamin S. Forward, Bocabec (CA)

Correspondence Address: KLARQUIST SPARKMAN, LLP, 121 SW SALMON STREET, SUITE 1600, PORTLAND, OR, 97204, US

(73) Assignee: University of Victoria Innovation and Development Corporation

(21) Appl. No.: 10/235,113

(22) Filed: Sep. 4, 2002

Related U.S. Application Data
(63) Continuation-in-part of application No. 10/117,641, filed on Apr. 3, 2002.
Application No. 10/117,641 is a division of application No. 09/632,538, filed on Aug. 4, 2000, now Pat. No. 6,440,674.

(51) Int. Cl. A01H 5/00; C12P 21/02; C07K 14/415; C12N 5/04; C07H 21/04; C12N 15/82

(52) U.S. Cl. 536/23.6; 800/288; 530/370; 435/69.1; 435/320.1; 435/419

(57) ABSTRACT

Disclosed is a luminal binding protein promoter (PmBiPPro1) including deletions, fusions, and variants thereof. The promoter can be used to direct expression of transgenes.

### CROSS-REFERENCE TO RELATED APPLICATION

This is a continuation-in-part of Application No. 10/117,641 filed Apr. 3, 2002, which is a division of application Ser. No. 09/632,538 filed Aug. 4, 2000, now Patent No. 6,440,674, all herein incorporated by reference in their entirety.

### FIELD

This disclosure relates to an isolated luminal binding protein promoter sequence and methods for its use.

### BACKGROUND

Luminal binding proteins (BiP) have been identified as a type of molecular chaperone localized within the endoplasmic reticulum (ER) and nuclear envelope of eukaryotic cells. BiP is a member of the heat-shock protein 70 (HSP70) family of proteins (Haas, Experimentia 50:1012-20, 1994). BiP assists in the co-translational translocation of newly synthesized polypeptides across the ER membrane in yeast (Vogel et al., J. Cell Biol. 110:1885-95, 1990; and Nguyen et al., Proc. Natl. Acad. Sci. USA 88:1565-9, 1991). BiP remains associated with polypeptides until they attain their properly folded conformation and/or subunit assembly. For polypeptides that are unable to attain their mature conformation due to misfolding (Scbmitz et al., EMBO J. 14:1091-8, 1995) or lack of a subunit component (Knittler et al., Proc. Natl. Acad. Sci. USA 92:1764-8, 1995), BiP remains associated with the polypeptide until the polypeptide is degraded.

In angiosperms, the expression of BiP is subject to developmental, hormonal, stress-induced, and diurnal regulation (Denecke et al., Plant Cell 3:1025-35, 1991; Jones et al., Plant Physiol. 97:456-9, 1991; Anderson et al., Plant Physiol. 104:1359-70, 1994; Kalinski et al., Planta 195:611-21, 1995; and Figueiredo et al., Braz. J. Plant Physiol. 9:103-10, 1997). BiP associates with the bean storage protein phaseolin (D'Amico et al., Plant J. 2:443-55, 1992; Pedrazzini et al., Plant J. 5:103-110, 1994) and with rice prolamines (Li et al., Science 262:1054-6, 1993). High levels of BiP expression are associated with the accumulation of protein intermediates that are unable to attain their proper folded conformation because of mutations, such as those seen in the maize zein regulatory mutants “floury-2,” “defective endosperm-B30,” and “mucronate” (Boston et al., Plant Cell 3:497-505, 1991; Fontes et al., Plant Cell 3:483-96, 1991).

Treatment with tunicamycin, which inhibits N-linked glycosylation and proper protein folding, also results in increased levels of BiP expression (Denecke et al., Plant Cell 3:1025-35, 1991; and D'Amico et al., Plant J. 2:443-455, 1992). However, the increased expression resulting from unfolded proteins and from increased levels of secretory protein traffic may be mediated through different signals (Pahl et al., EMBO J. 14:2580-8, 1995).

### SUMMARY

The present disclosure provides a Douglas-fir (Pseudotsuga menziesii) luminal binding protein promoter (PmBiPPro1; SEQ ID NO: 31). Expression of a PmBiP protein (SEQ ID NO: 36) is developmentally-regulated and inducible by environmental changes. The PmBiPPro1 promoter, and fragments and variants thereof, are useful for expressing heterologous proteins, for example transiently in host cells or transgenically in stably transformed cells and plants.

One aspect of the disclosure provides a PmBiP promoter, fragments, deletions, fusions, and variants thereof. The variant promoters are characterized by their retention of at least 50%, 60%, 70%, 80%, 90%, 95%, 98%, or even 99% sequence identity with the disclosed promoter sequences (SEQ ID NOS: 16, 17, 18, and 31), or by retention of at least 20, 30, 40, 50, 60, 100, 125, 150, 175, 200, 250, 275, 300 or even 500, consecutive nucleic acid residues of the disclosed promoter sequences (SEQ ID NOS: 16, 17, 18, and 31). Variants, fragments, deletions, and fusions of SEQ ID NOS: 16, 17, 18, and 31 retain promoter activity, such as native PmBiP promoter activity.

Other promoters, such as the CaMV35S promoter, can be altered through the introduction of sequences found in the PmBiP promoter (SEQ ID NO: 31). The resulting promoter is also characterized by its retention of at least 20, 30, 40, 50, or 60 consecutive nucleic acid residues of the disclosed promoter sequences (SEQ ID NOS: 16, 17, 18, and 31).

An alternative method of characterizing promoters is by analyzing promoter elements found within a promoter sequence. Hence, the disclosure also provides promoters that maintain promoter activity, such as PmBiP promoter activity, and include at least 8 promoter elements selected from one or more of (such as two or more) of the group consisting of the E-box motif (SEQ ID NO: 1), the ACGT-core element (SEQ ID NO: 4), the CAAT-box (SEQ ID NO: 9), the CANABNNAPA element (SEQ ID NO: 12), the HEXMOTIF element (SEQ ID NO: 27), the MNF1 element (SEQ ID NO: 28), the POLLENILELAT52 element (SEQ ID NO: 29), the ROOTMOTIF element (SEQ ID NO: 30), the 2SSEEDPROTBANAP element (SEQ ID NO: 32), the BOXIIPCCHS element (SEQ ID NO: 33), the ASF1MOTIF element (SEQ ID NO: 34), the UPRE element (SEQ ID NO: 42), wherein at least one of the at least 8 promoter elements is a UPRE element and the promoter displays promoter activity. In particular examples, at least one of the at least 8 promoter elements is a BOXIIPCCHS element (SEQ ID NO: 33), an ASF1MOTIF element (SEQ ID NO: 34), a QAR element (SEQ ID NO: 41), a NRR element (SEQ ID NO: 40), and/or an LTRE element (SEQ ID NOS: 38 and 39). In some examples, the isolated promoter elements are further selected from the group consisting of LTRE elements (SEQ ID NOS: 38 and 39), NRR elements (SEQ ID NO: 40), and QAR elements (SEQ ID NO: 41).

The disclosure also provides promoters that contain the following promoter elements in the following orientation: 5′-ACGT-core element (SEQ ID NO: 4), E-box motif (SEQ ID NO: 1), CAAT-box (SEQ ID NO: 9); 2SSEEDPROTBANAP element (SEQ ID NO: 32) or CANABNNAPA element (SEQ ID NO: 12); HEXMOTIF element (SEQ ID NO: 27), CAAT-box (SEQ ID NO: 9); UPRE element (SEQ ID NO: 42); E-box motif (SEQ ID NO: 1), ASF1MOTIF element (SEQ ID NO: 34), POLLEN1LELAT52 element (SEQ ID NO: 29), and MNF1 element (SEQ ID NO: 28)-3′.

Another aspect of the disclosure provides vectors containing the disclosed promoters and variants thereof. The vectors can be transformed into host cells, such as plant cells. If the host cell is a plant cell, the transformed host cell can give rise to a transgenic plant, such as transgenic maize; wheat; rice; millet; tobacco; sorghum; rye; barley; brassica; seaweeds; lemna; oat; soybean; cotton; legumes; rape/canola; alfalfa; flax; peanut; clover; cucurbits; cassaya; potato; vegetables such as carrot, radish, pea, lentil, cabbage, lettuce, tomato, cauliflower, broccoli, Brussel sprouts, peppers; fruit trees such as apple, pear, peach, and apricot trees; nut trees such as walnut and filbert tress; flowers such as orchids, carnations, sunflower, safflower, and roses; cacao; deciduous trees such as poplar and elms; conifers such as Douglas-fir and spruce; turf grasses; rubber trees; and members of the genus Hevea.

The disclosure also provides transgenes. These transgenes include a PmBiP promoter sequence, operably linked to one or more open reading frames (ORFs). The transgenes can be cloned into vectors and subsequently used to transform host cells such as bacterial, insect, mammalian, fungal, yeast, or plant cells.

The disclosure also provides methods for expressing proteins in host cells, such as plant host cells. Such methods involve operably linking a disclosed promoter, such as a PmBiP promoter, to at least one ORF to produce a transgene, and introducing the transgene into a plant. Accordingly, the disclosure also provides proteins produced by these methods.

PmBiP promoters are inducible by wounding and cold temperatures, such as temperatures below about 20° C., such as below about 10° C., such as below about 4° C. The amount of mRNA encoding PmBiP protein and the BiP protein itself is increased at cold temperatures, thus making the PmBiP promoter useful for expressing proteins. This is because cold temperatures serve to stabilize the protein during translation. Accordingly, another aspect of the disclosure provides inducible promoters derived through the use of fragments of the PmBiP promoters described herein.

These and other aspects of the disclosure will become readily apparent from the following detailed description.

### BRIEF DESCRIPTION OF THE FIGURES

FIG. 1 is a bar graph depicting the expression of PmBiP RNA during Douglas-fir seed development.

FIG. 2 is a bar graph showing the expression of PmBiP RNA during germination and early seedling development.

FIG. 3 is a bar graph showing changes in PmBiP RNA levels in response to cold treatment.

FIGS. 4A and 4B are graphs showing the seasonal variation of PmBiP protein in needles from 1-year-old seedlings. (A) PmBiP protein levels. (B) Monthly average temperatures (° C.).

FIGS. 5A-5I show a table of cis-acting elements located in the PmBiP promoter (SEQ ID NO: 31) using the plant cis-acting regulatory database (PLACE; Higo et al., Plant Mol. Biol. Rep. 5:387-405. 1987; and Prestridge, CABIOS 7:203-6, 1991). Cis elements are grouped according to type. Elements deleted from PmBiPPro1-1 construct to form PmBiPPro1-3 are darkly shaded, elements deleted from PmBiPPro1-3 construct to form PmBiPPro1-5 are lightly shaded, and elements remaining in PmBiPPro1-5 are not shaded.

FIG. 6A is a schematic diagram showing a comparison of the full-length PmBiPPro1sequence (SEQ ID NO: 31; PmBiPPro1) to the deletion constructs PmBiPPro1-1 (SEQ ID NO: 16), PmBiPPro1-3 (SEQ ID NO: 17), and PmBiPPro1-5 (SEQ ID NO: 18).

FIG. 6B shows the sequence of PmBiPPro1 numbered from 5′ to 3′ with predicted promoter regions shown. Numbering is not relative to the transcriptional start site.

FIG. 6C shows the alignment of a yeast UPRE (SEQ ID NO: 43) and the PmBiPPro1 putative UPRE (SEQ ID NO: 42). Critical residues for yeast UPRE function are boxed or underlined. Boxed residues represent partial palindromic half sites separated by a single nucleotide spacer. Asterisks indicate identical residues.

FIG. 7 is a bar graph showing the effect of transient expression of GUS in Douglas-fir zygotic embryos under the control of the CaMV35S, PmBiPPro1-1 (SEQ ID NO: 16), PmBiPPro1-3 (SEQ ID NO: 17), and PmBiPPro1-5 (SEQ ID NO: 18) promoter sequences.

FIG. 8 is a bar graph showing the result of in vitro GUS activity of 19-day-old transgenic Arabidopsis plants containing various PmBiPPro1 constructs. Two transformants were examined for each construct.

FIG. 9 is a bar graph showing PmBiPPro1-1 (SEQ ID NO: 16) expression in response to wounding in 21-day-old Arabidopsis cotyledons.

FIG. 10 is the nucleotide (SEQ ID NO. 35) and deduced amino acid sequence (SEQ ID NO. 36) of PmBiP cDNA. The nucleotide sequence is numbered on the left, and the amino acid sequence is numbered on the right. Untranslated regions are in lower-case letters and the open reading frame is capitalized. The three potential start codons are underlined, with the amino acid sequence beginning at the third codon. The predicted signal-peptide cleavage site and beginning of the mature PmBiP amino acid sequence is indicated by an asterisk (Nielsen et al., Protein Eng. 10:1-6, 1997). The ER-retention signal sequence is boxed. The 13 carboxy-terminal amino acids used to generate an antiserum to the peptide are indicated in bold italics.

### SEQUENCE LISTING

The nucleic and amino acid sequences listed in the accompanying sequence listing are shown using standard letter abbreviations for nucleotide bases, and three-letter code for amino acids. Only one strand of each nucleic acid sequence is shown, but the complementary strand is understood to be included by any reference to the displayed strand.

SEQ ID NO: 1 is the nucleic acid sequence of an E-box motif.

SEQ ID NO: 2 is the nucleic acid sequence of a RY-repeated element.

SEQ ID NO: 3 is the nucleic acid sequence of an AT-rich region.

SEQ ID NO: 4 is the nucleic acid sequence of an ACGT-core element.

SEQ ID NO: 5 is the nucleic acid sequence of an opaque-2-like binding site.

SEQ ID NOs: 6 and 7 are the nucleic acid sequences of respective conserved gymnosperm-like regions.

SEQ ID NO: 8 is a nucleic acid sequence of a TATA box.

SEQ ID NO: 9 is the nucleic acid sequence of a CAAT box.

SEQ ID NO: 10 is the nucleic acid sequence of a MYBPZM element.

SEQ ID NO: 11 is the nucleic acid sequence of a GTI consensus sequence.

SEQ ID NO: 12 is the is the nucleic acid sequence of a CANBNAPA element.

SEQ ID NO: 13 is the nucleic acid sequence of a MARARS element.

SEQ ID NOS: 14 and 15 are specific examples of opaque-2-like binding sites.

SEQ ID NO: 16 is the nucleic acid sequence of the PmBiPPro1-1 promoter construct.

SEQ ID NO: 17 is the nucleic acid sequence of the PmBiPPro1-3 construct.

SEQ ID NO: 18 is the nucleic acid sequence of the PmBiPPro1-5 construct.

SEQ ID NOS: 19-22 are PCR primers used in inverse-PCR reactions.

SEQ ID NOS: 23-26 are PCR primers used to clone the PmBiP promoter.

SEQ ID NO: 27 is the nucleic acid sequence of a HEXMOTIF element.

SEQ ID NO: 28 is the nucleic acid sequence of a MNF1 element.

SEQ ID NO: 29 is the nucleic acid sequence of a POLLEN1LELAT52 element.

SEQ ID NO: 30 is the nucleic acid sequence of a ROOTMOTIF element.

SEQ ID NO: 31 is the complete PmBiP promoter sequence.

SEQ ID NO: 32 is the nucleic acid sequence of a 2SSEEDPROTBANAP element.

SEQ ID NO: 33 is the nucleic acid sequence of a BOXIIPCCHS element.

SEQ ID NO: 34 is the nucleic acid sequence of an ASF1MOTIF.

SEQ ID NO: 35 is the cDNA sequence encoding the luminal binding protein BiP.

SEQ ID NO: 36 is the amino acid sequence of the luminal binding protein.

SEQ ID NO: 37 is the amino acid sequence of the endoplasmic reticulum (ER) retention sequence, HEEL.

SEQ ID NOS: 38 and 39 are nucleic acid sequences of a low-temperature-responsive element (LTRE).

SEQ ID NO: 40 is a nucleic acid sequence of a negative regulatory region (NRR) element.

SEQ ID NO: 41 is a nucleic acid sequence of a quantitative activator region (QAR) element.

SEQ ID NO: 42 is a nucleic acid sequence of an unfolded protein response element (UPRE).

SEQ ID NO: 43 is a nucleic acid sequence of a yeast UPRE.

### DETAILED DESCRIPTION DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein, “comprising” means” “including” and the singular forms “a” or “an” or “the” include plural references unless the context clearly dictates otherwise. For example, reference to “comprising a promoter” includes one or a plurality of such promoter, and reference to “comprising the cell” includes reference to one or more cells and equivalents thereof known to those skilled in the art, and so forth.

Unless explained otherwise, all technical and scientific terms used herein have the same meaning as commonly understood to one of ordinary skill in the art to which this disclosure belongs. Definitions of common terms in molecular biology may be found in Lewin, Genes VII, Oxford University Press, 1999 (ISBN 0-19-879276-X); Kendrew et al. (eds.), The Encyclopedia of Molecular Biology, Blackwell Science, Ltd., 1994 (ISBN 0-632-02182-9); and Meyers (ed.), Molecular Biology and Biotechnology: A Comprehensive Desk Reference, VCH Publishers, Inc., 1995 (ISBN 1-56081-569-8). The materials, methods, and examples are illustrative only and not intended to be limiting. Other features and advantages of the disclosure will be apparent from the following detailed description, and from the claims.

cDNA (complementary DNA): A piece of DNA lacking internal, non-coding segments (introns) and transcriptional regulatory sequences. cDNA also may contain untranslated regions (UTRs) that are responsible for translational control in the corresponding RNA molecule. cDNA usually is synthesized in the laboratory by reverse transcription from messenger RNA extracted from cells.

Cationic Peptides: Endogenous antimicrobial peptides produced by plants and animals typically consisting of about 12-45 amino acids. Additionally, they are amphipathic molecules having a net positive charge (cationic) at physiological pH. Although cationic antimicrobial peptides (CAPs) are structurally diverse, they fall into two general classes of structures: α-helical peptides, such as the cecropins and magainans, and β-sheet peptides stabilized by intramolecular disulphide bonds, such as the defensins, protegrins, and tachyplesins. Hancock and Lehrer, Trends Biotechnol. 16:82-8, 1998; Zasloff, Curr. Opin. Immunol. 4:3-7, 1992; Cociancich et al., Biochem. J. 300:567-575 1994; and Piers and Hancock, Mol. Microbiol. 12:951-8, 1994. Natural CAPs vary greatly in their respective spectra of biological activities, including killing bacteria (Gram-positive and -negative), fungi, protozoa, and viruses. CAPs normally kill susceptible microorganisms in vitro at concentrations from about 0.25 μg/ml to 4 μg/ml (Hancock and Lehrer, Trends Biotechnol. 16:82-8, 1998), providing exciting possibilities in the face of the declining efficacy of conventional antibiotics. Furthermore, the expression of CAP in plants may introduce broad-spectrum resistance to phytopathogenic microorganisms. Jaynes, Plant Science 89:43-53, 1993; and Misra and Zhang, Plant Physiol. 106:977-81, 1994.

Cationic peptides can be expressed under the control of the disclosed PmBiP promoter and fragments and variants thereof. Other proteins that confer disease resistance, resistance to environmental stress, resistance to insect infestation, or herbicide resistance, or alter consumer-related characteristics such as shelf-life, color, or nutritional value, also may be expressed under the control of the PmBiP promoter described herein.

Comprises: A term that means “including.” For example, “comprising A or B” means including A or B, or both A and B, unless clearly indicated otherwise.

Conservative substitution: One or more amino acid substitutions (for example 2, 5 or 10 residues) for amino acid residues having similar biochemical properties. Typically, conservative substitutions have little to no impact on the activity of a resulting polypeptide. For example, ideally, a PmBiP peptide including one or more conservative substitutions retains PmBiP activity. A polypeptide can be produced to contain one or more conservative substitutions by manipulating the nucleotide sequence that encodes that polypeptide using, for example, standard procedures such as site-directed mutagenesis or PCR.

Substitutional variants are those in which at least one residue in the amino acid sequence has been removed and a different residue inserted in its place. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gin; Asp for Glu; Pro for Gly; Asn or Gin for His; Leu or Val for Ile; Ile or Val for Leu; Arg or Gin for Lys; Leu or Ile for Met; Met, Leu or Tyr for Phe; Thr for Ser; Ser for Thr; Tyr for Trp; Trp or Phe for Tyr; and Ile or Leu for Val.

Further information about conservative substitutions can be found in, among other locations in, Ben-Bassat et al., (J. Bacteriol. 169:751-7, 1987), O'Reagan et al., (Gene 77:237-51, 1989), Sahin-Toth et al., (Protein Sci. 3:240-7, 1994), Hochuli et al., (Bio/Technotogy 6:1321-5, 1988), WO 00/67796 (Curd et al.) and in standard textbooks of genetics and molecular biology.

In one example, such variants can be readily selected for additional testing by performing an assay to determine if the variant retains PmBiP activity.

Deletion: Removal of one or more nucleic acid residues from a DNA sequence, or one or more amino acid residues from a protein sequence, the regions on either side of the removed sequence being joined together.

Douglas-fir luminal binding protein promoter (PmBiPPro1). The nucleic acid sequence of a PmBiP promoter is provided in SEQ ID NO: 31. However, the disclosure also encompasses variants, fusions, and fragments of the PmBiP promoter that are characterized by their ability to maintain promoter activity, at a minimum, and in some embodiments maintain native PmBiP promoter activity. Variants retain at least 50%, 60%, 70%, 80%, 90%, 95%, 98% or even 99% sequence identity when compared to the nucleic acid sequences shown in SEQ ID NOS: 16, 17, 18, and 31. Variants can be isolated from nature using the hybridization or PCR techniques described below, or they can be made by manipulating the nucleic acid sequences shown in SEQ ID NOS: 16, 17, 18, and 31 using standard molecular biology methods.

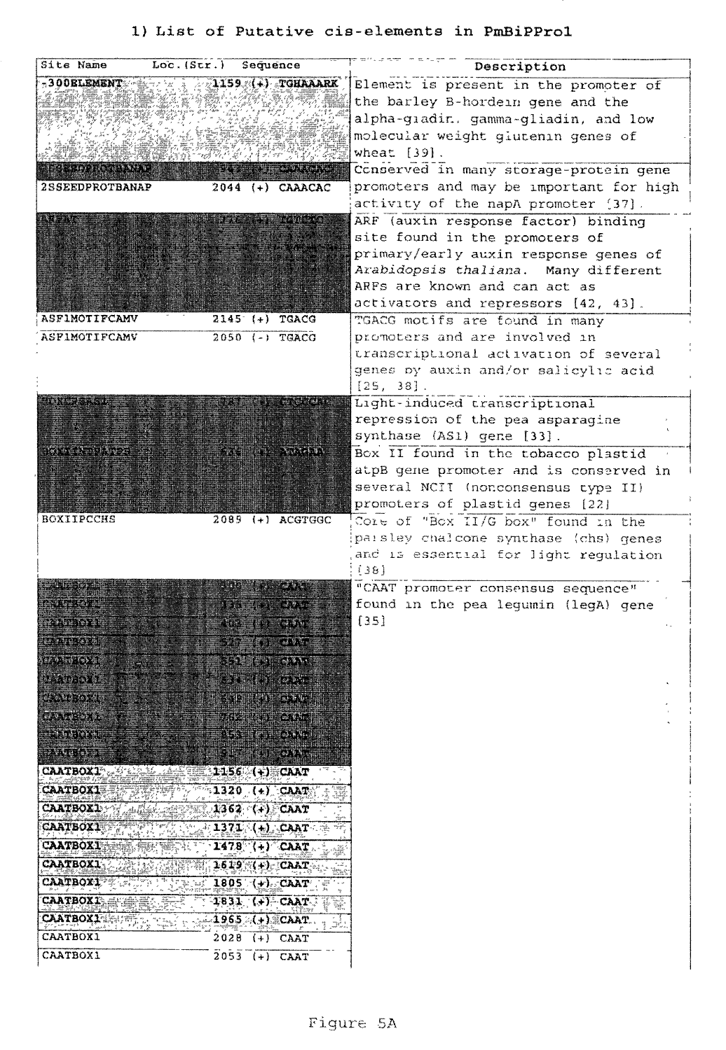

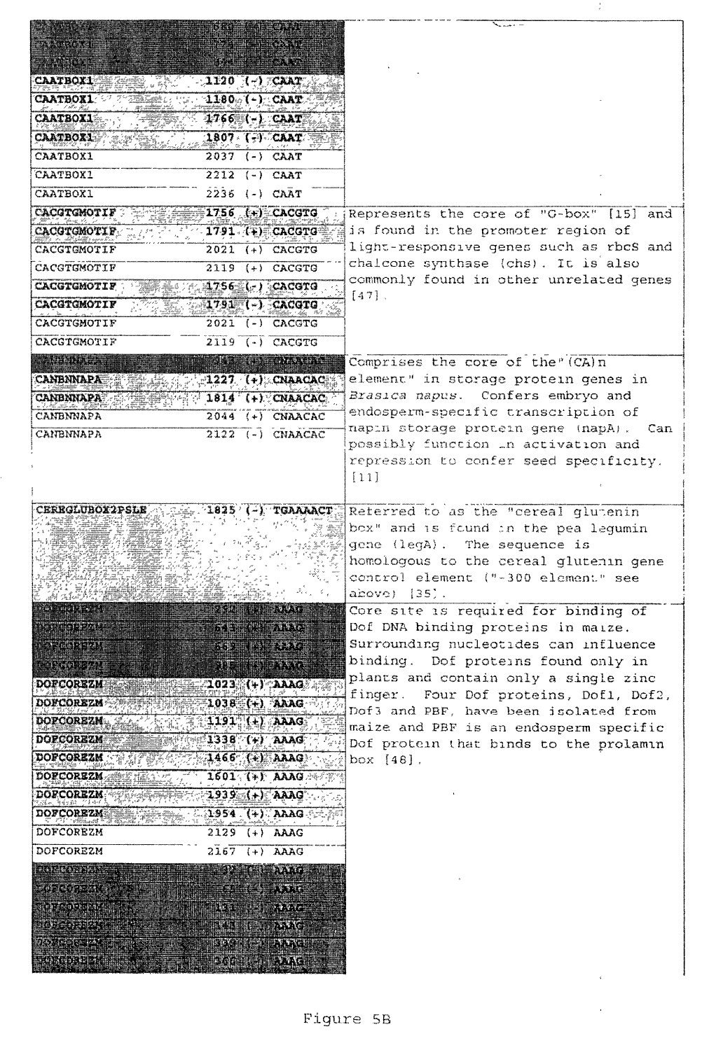

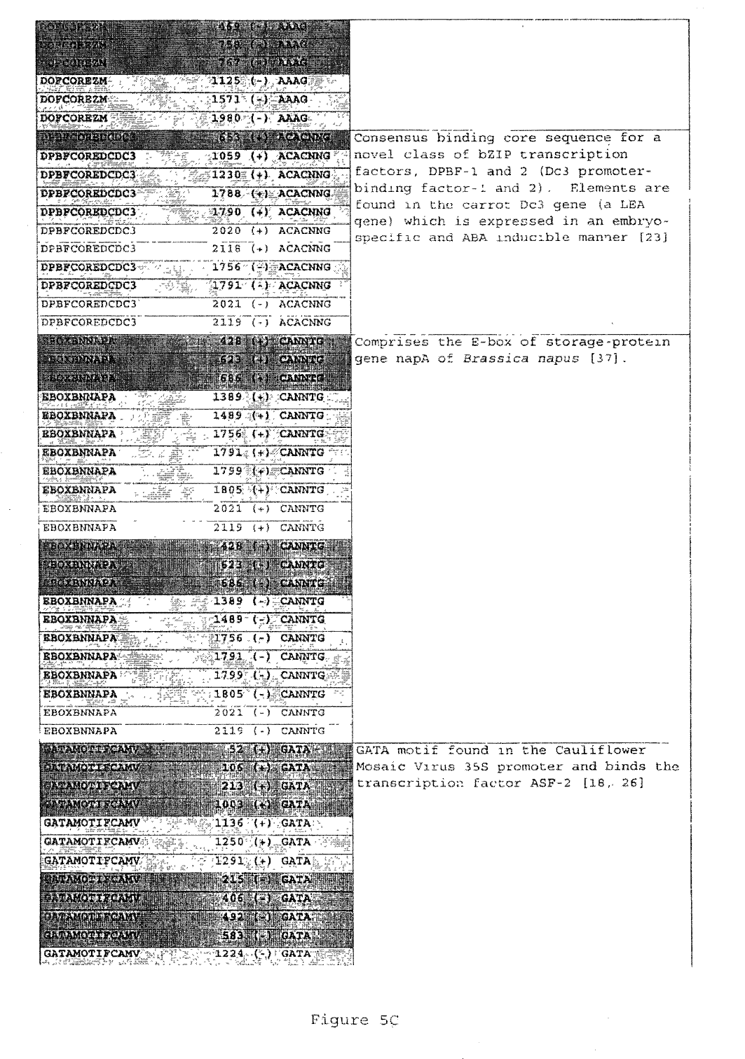

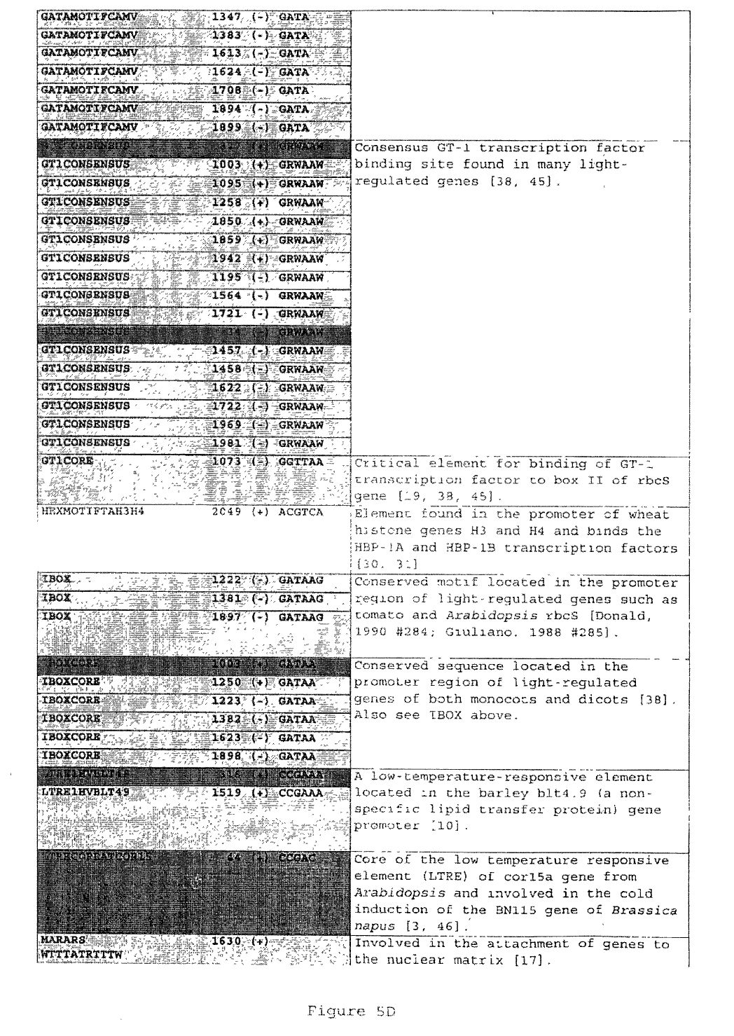

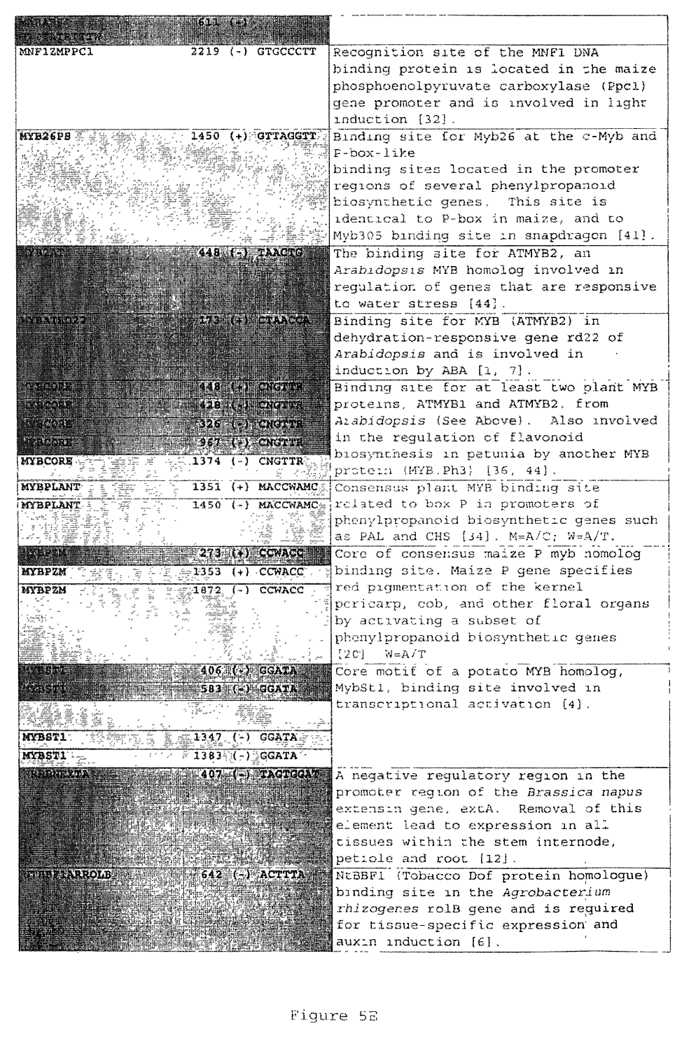

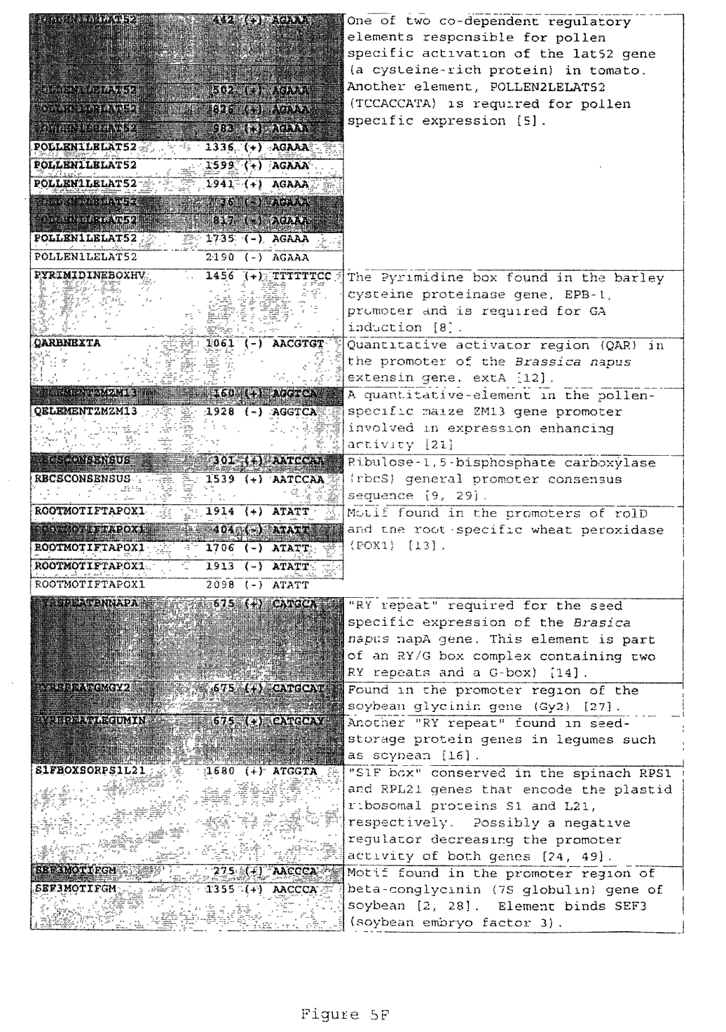

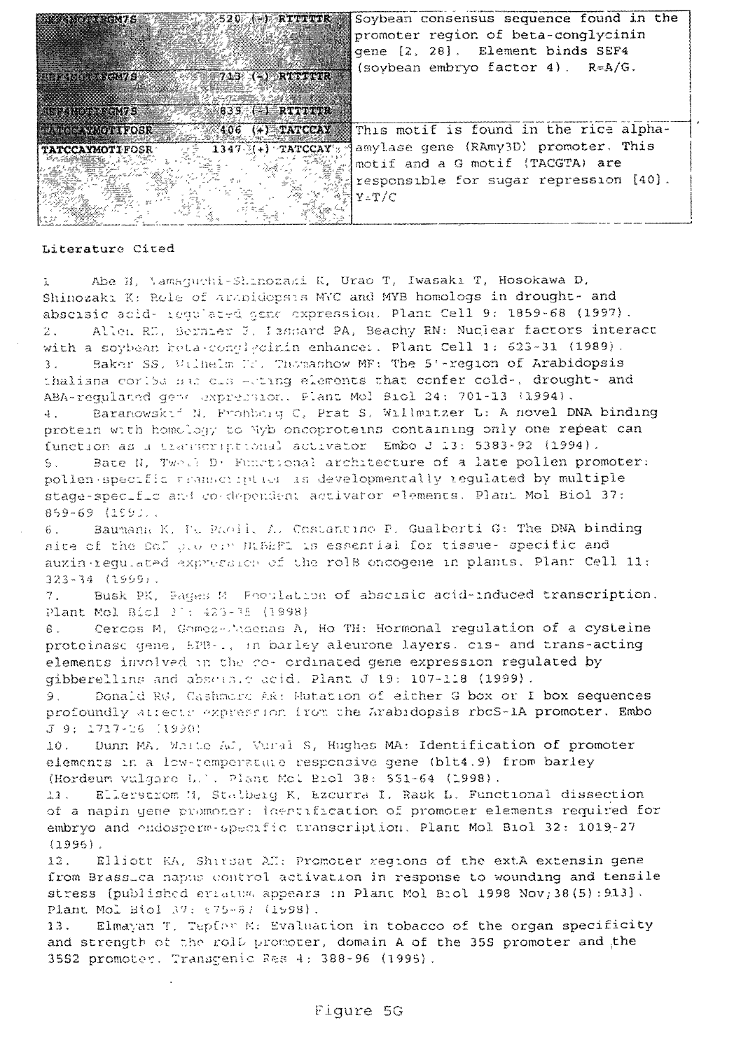

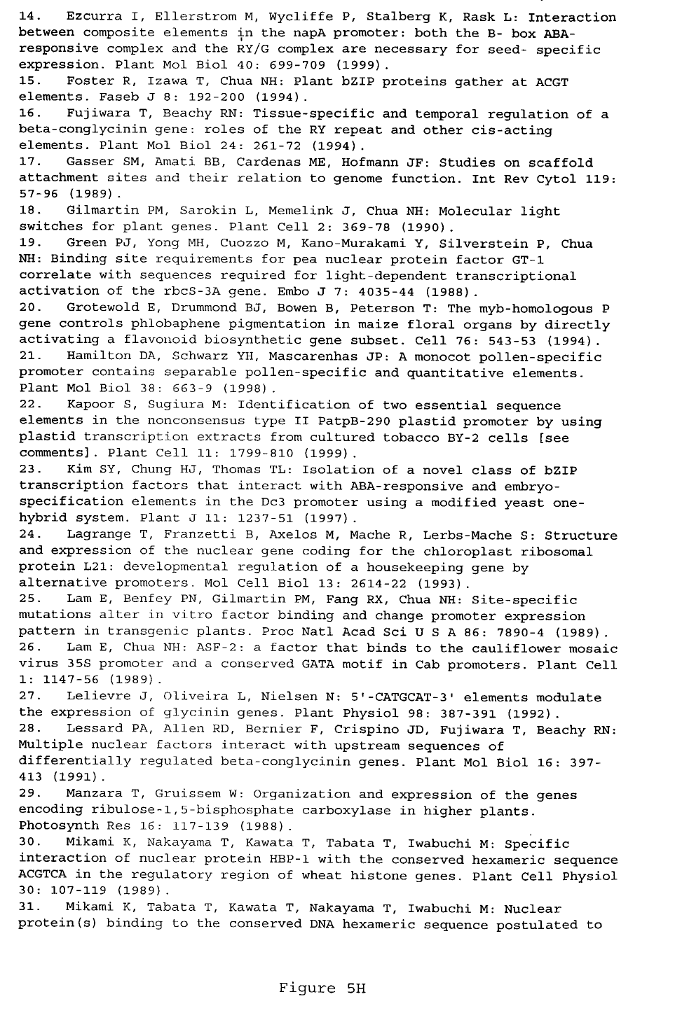

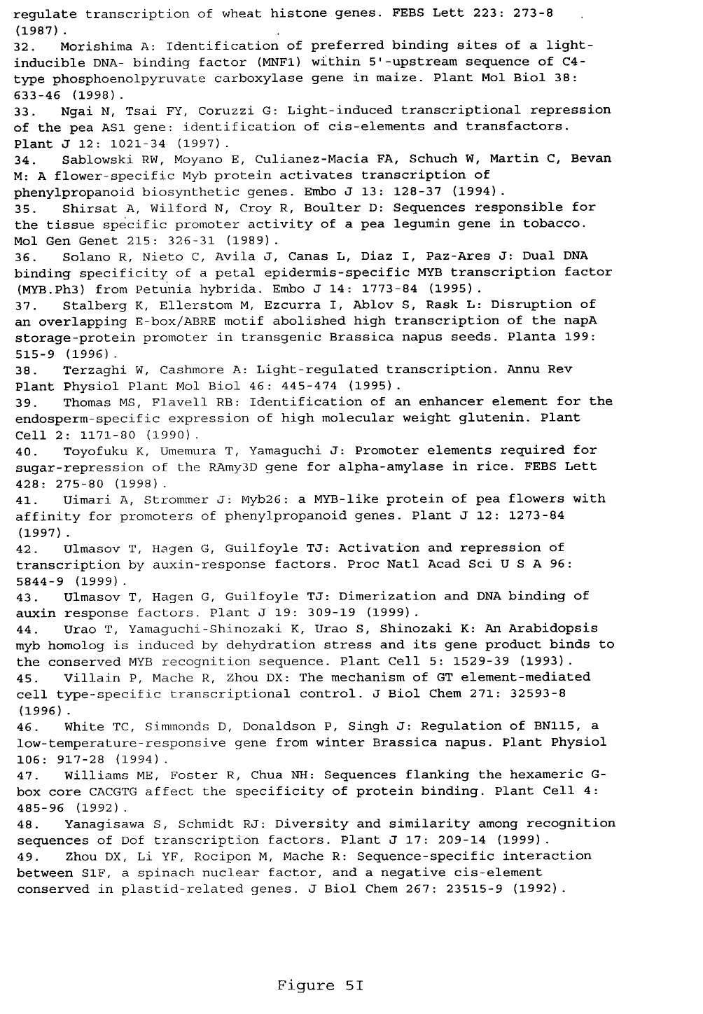

The PmBiP promoter shown in SEQ ID NO: 31 contains several distinct promoter elements and inter-element spaces that are arranged in series in the DNA fragment (see FIGS. 5A-G). One or more of these elements or inter-element spaces can be altered, deleted, and/or duplicated without loss of promoter activity. One of ordinary skill in the art will appreciate that other promoter elements can be added to a PmBiP promoter without loss of promoter activity and/or native PmBiP promoter activity. Hence, the disclosure provides promoters that maintain native promoter activity and/or promoter activity and include at least 8, 10, 11, 12, 14, 16, 18, 20, 22, 30, or 35 of the promoter elements contained within the PmBiP promoter (SEQ ID NO: 31).

Variants of the PmBiP promoter also can be characterized by the number of contiguous nucleic acid residues they share with the PmBiP promoter, such as those shown in SEQ ID NOS: 16, 17, 18, and 31. For example, a variant of the PmBiP promoter can share at least 20, 25, 30, 40, 50, 60, 100, 150, 200, 250, 300, or 500 contiguous nucleic acid residues with SEQ ID NOS: 16, 17, 18, and 31. Such variants additionally will be characterized by their ability to drive the expression of a transgene operably linked to it.

Exogenous: The term “exogenous” as used herein with reference to nucleic acid and a particular cell refers to any nucleic acid that does not originate from that particular cell as found in nature. Thus, a non-naturally-occurring nucleic acid is considered to be exogenous to a cell once introduced into the cell. Nucleic acid that is naturally-occurring also can be exogenous to a particular cell. For example, an entire chromosome isolated from a cell of plant X is an exogenous nucleic acid with respect to a cell of plant Y once that chromosome is introduced into Y's cell.

Functional deletion: A mutation, partial or complete deletion, insertion, or other variation made to a gene sequence which inhibits production of the gene product, and/or renders the gene product non-functional.

Hybridization: A method of testing for complementarity in the nucleotide sequence of two nucleic acid molecules, based on the ability of complementary single-stranded DNA and/or RNA to form a duplex molecule. Nucleic acid hybridization techniques can be used to obtain an isolated nucleic acid within the scope of the disclosure. Briefly, any nucleic acid having some homology to a PmBiP promoter (such as homology to SEQ ID NO: 31 or variants or fragments thereof) can be used as a probe to identify a similar nucleic acid by hybridization under conditions of moderate to high stringency. Once identified, the nucleic acid then can be purified, sequenced, and analyzed to determine if it is a PmBiP promoter having PmBiP promoter activity.

Hybridization can be done by Southern or Northern analysis to identify a DNA or RNA sequence, respectively, that hybridizes to a probe. The probe can be labeled, for example with a biotin, a fluorophore, digoxygenin, an enzyme, or a radioisotope such as ³²P. The DNA or RNA to be analyzed can be electrophoretically separated on an agarose or polyacrylamide gel, transferred to nitrocellulose, nylon, or other suitable membrane, and hybridized with the probe using standard techniques well known in the art such as those described in sections 7.39-7.52 of Sambrook et al., (1989) Molecular Cloning, second edition, Cold Spring Harbor Laboratory, Plainview, N.Y. Typically, a probe is at least about 20 nucleotides in length. For example, a probe including 20 contiguous nucleotides of a PmBiP promoter (such as 20 contiguous nucleotides of SEQ ID NO: 31, or 16-18) can be used to identify an identical or similar nucleic acid. In addition, probes longer or shorter than 20 nucleotides can be used.

The disclosure also provides isolated nucleic acid sequences that are at least about 12 bases in length (e.g., at least about 13, 14, 15, 16, 17, 18, 19, 20, 25, 30, 40, 50, 60, 100, 250, 500, 750, 1000, 1500, 2000, or 3000 bases in length) and hybridize, under hybridization conditions, to the sense or antisense strand of an alanine PmBiP promoter nucleic acid sequence, for example SEQ ID NO: 31). The hybridization conditions can be moderately or highly stringent hybridization conditions.

Moderately stringent hybridization conditions are when the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2×SSC and 0.1% sodium dodecyl sulfate.

Highly stringent hybridization conditions are when the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

Insertion: The addition of one or more nucleotide or amino acid residues into a nucleic acid or amino acid sequence, respectively.

Isolated: An “isolated” biological component (such as a nucleic acid, protein, or organelle) has been substantially separated or purified from other biological components in the cell of the organism in which the component naturally occurs, i.e., other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

In one example, isolated refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

In one example, the term “isolated” as used with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

Native PmBiP Promoter Activity: Native PmBiP promoter activity is characterized by developmental-specific transcription. mRNA encoding the PmBiP protein is expressed in seeds, following stratification and exposure to germination conditions, to a greater extent than in mature seeds. Hence, it is believed that the PmBiP promoter (SEQ ID NO: 31 or variants thereof, such as SEQ ID NOS: 16-18) will promote the expression of transgenes in a similar pattern. Developmental-specific activity is the ability of a promoter to promote transcription at a higher level during one stage in development when compared to another stage of development.

Furthermore, developmental-specific expression can be determined by creating transgenic plants and assaying the resulting transgenic tissues (e.g., leaves, flowers, seeds, roots) for transgene mRNA or by assaying for a reporter gene such as GUS. Developmental-specific expression is quantified by comparing the level of mRNA expressed in a tissue during one stage in development compared to the level expressed in the same tissue at another stage of development. The degree of developmental-specific expression is expressed in terms of a percentage of expression, i.e., the percentage of mRNA in one developmental stage compared to another. For example 100% (1×) expression denotes that an equal amount of expression is observed during two distinct stages of development, 200% (2×) denotes that twice as much mRNA is expressed in one tissue compared to another tissue. Native PmBiP promoter activity is the ability of the PmBiP promoter to drive the expression of mRNA to a greater degree during one stage in plant development compared to another stage in development (i.e., at least 101%). Of course, the PmBiP promoter (SEQ ID NO: 31) can show an even stronger bias for developmental-specific expression, such as at least 125%, 150%, 200%, 250%, or 300% developmental-specific expression in seeds.

In another or in an additional example, native PmBiP promoter activity is characterized by the ability of a PmPiP promoter or variant thereof to be induced in response to wounding and/or to promote gene expression at temperatures below 20° C., such as below 10° C.

Oligonucleotide (“oligo”): A linear polynucleotide sequence of up to about 100 nucleotide bases in length.

Open reading frame (ORF): A series of nucleotide triplets (codons) coding for amino acids without any internal termination codons. These sequences are usually translatable into a peptide.

Operably linked: A first nucleic acid sequence is “operably linked” with a second nucleic acid sequence whenever the first nucleic acid sequence is situated in a functional relationship with the second nucleic acid sequence. For instance, a promoter is operably linked to a coding sequence if the promoter affects the transcription or expression of the coding sequence, such as promotes transcription. Generally, operably linked DNA sequences are contiguous and, where necessary to join two protein-coding regions, are in the same reading frame.

Orthologs: Nucleic acid or amino acid sequences that share a common ancestral sequence, but that diverged when a species carrying that ancestral sequence split into two species. Orthologous sequences are usually also homologous sequences.

Polynucleotide: A linear nucleic acid sequence of any length. Therefore, a polynucleotide includes molecules which are at least about 15, 25, 50, 75, 100, 200 or 400 (oligonucleotides) and also nucleotides as long as a full-length cDNA.

Probes and primers: A “probe” includes an isolated nucleic acid containing a detectable label or reporter molecule. Labeled nucleic acid sequences can be used to identify other promoters and seed-storage proteins. Typical labels include radioactive isotopes, ligands, chemiluminescent agents, fluorophores, and enzymes. Methods for labeling and guidance in the choice of labels appropriate for various purposes are discussed in, for example, Sambrook et al. (ed.), Molecular Cloning: A Laboratory Manual 2nd ed., vol. 1-3, Cold Spring Harbor Laboratory Press, Cold Spring Harbor, N.Y., 1989, and Ausubel et al. (ed.) Current Protocols in Molecular Biology, Greene Publishing and Wiley-Interscience, New York (with periodic updates), 1987.

“Primers” are typically nucleic acid molecules having ten or more nucleotides (e.g., nucleic acid molecules having between about 10 nucleotides and about 100 nucleotides). A primer can be annealed to a complementary target nucleic acid strand by nucleic acid hybridization to form a hybrid between the primer and the target nucleic acid strand, and then extended along the target nucleic acid strand by, for example, a DNA polymerase enzyme. Primer pairs can be used for amplification of a nucleic acid sequence, for example, by the polymerase chain reaction (PCR) or other nucleic-acid amplification methods known in the art.

Methods for preparing and using probes and primers are described, for example, in references such as Sambrook et al. (ed.), Molecular Cloning: A Laboratory Manual, 2nd ed., vol. 1-3, Cold Spring Harbor Laboratory Press, Cold Spring Harbor, N.Y., 1989; Ausubel et al. (ed.), Current Protocols in Molecular Biology, Greene Publishing and Wiley-Interscience, New York (with periodic updates), 1987; and Innis et al., PCR Protocols: A Guide to Methods and Applications, Academic Press: San Diego, 1990. PCR primer pairs can be derived from a known sequence, for example, by using computer programs intended for that purpose such as Primer (Version 0.5, © 1991, Whitehead Institute for Biomedical Research, Cambridge, Mass.). One of skill in the art will appreciate that the specificity of a particular probe or primer increases with the length, but that a probe or primer can range in size from a full-length sequence to sequences as short as five consecutive nucleotides. Thus, for example, a primer of 20 consecutive nucleotides can anneal to a target with a higher specificity than a corresponding primer of only 15 nucleotides. Thus, in order to obtain greater specificity, probes and primers can be selected that comprise, for example, 10, 20, 25, 30, 35, 40, 50, 60, 70, 80, 90, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 2000, 2050, 2100, 2150, 2200, 2250, 2300, 2350, 2400, 2450, 2500, 2550, 2600, 2650, 2700, 2750, 2800, 2850, 2900, 3000 or more consecutive nucleotides.

Promoter: An array of nucleic acid control sequences which direct transcription of a nucleic acid. A promoter includes necessary nucleic acid sequences near the start site of transcription, such as, in the case of a polymerase 11 type promoter, a TATA element. A promoter also optionally includes distal enhancer or repressor elements which can be located as much as several thousand base pairs from the start site of transcription.

Promoter Activity: The ability of a DNA sequence to promote or enhance transcription. Promoter activity varies with the number and position of the promoter elements. For example, a PmBiP promoter can be altered to remove its developmental-specific activity (native activity) without loss of its ability to promote transcription.

Promoter elements: Sub-domains within the promoter that confer tissue-specific expression, enhance expression, or inhibit expression. A promoter can contain multiple promoter elements. Furthermore, some elements can appear more than once within a single promoter. Examples of such elements are E-box motifs (SEQ ID NO: 1), RY-repeat elements (SEQ ID NO: 2), AT-rich regions (SEQ ID NO: 3), ACGT-core elements (SEQ ID NO: 4), Opaque-2-like elements (SEQ ID NO: 5), conserved gymnosperm-like regions (SEQ ID NOS: 6 and 7), CAAT-boxes (SEQ ID NO: 9), CANABNNAPA elements (SEQ ID NO: 12), HEXMOTIF elements (SEQ ID NO: 27), MNF1 elements (SEQ ID NO: 28), POLLENI LELAT52 elements (SEQ ID NO: 29), ROOTMOTIF elements (SEQ ID NO: 30), 2SSEEDPROTBANAP elements (SEQ ID NO: 32), BOXIIPCCHS elements (SEQ ID NO: 33), ASF1MOTIF elements (SEQ ID NO: 34), UPRE elements (SEQ ID NO: 42), LTRE elements (SEQ ID NOS: 38 and 39), NRR elements (SEQ ID NO: 40), and QAR elements (SEQ ID NO: 41). Additional examples of promoter elements can be found in U.S. Pat. No. 5,723,751 to Chua; U.S. Pat. No. 5,608,149 to Barry et al.; U.S. Pat. No. 5,589,615 to De Clercq et al.; U.S. Pat. No. 5,589,583 to Klee et al.; U.S. Pat. No. 5,677,474 to Rogers; U.S. Pat. No. 5,487,991 to Vandekerckhove et al.; and U.S. Pat. No. 5,530,194 to Knauf et al. Typically, a TATA box is found on the 3′-end of the series of promoter elements.

Examples of specific promoter elements are provided above and in the sequence listing. However, one of skill in the art will appreciate that the specific examples shown in the sequence listing can be modified while still maintaining activity. For example a base in an RY-repeat element can be altered by the substitution of one or more acid residues without the RY-repeat element losing its functionality within the overall promoter sequence.

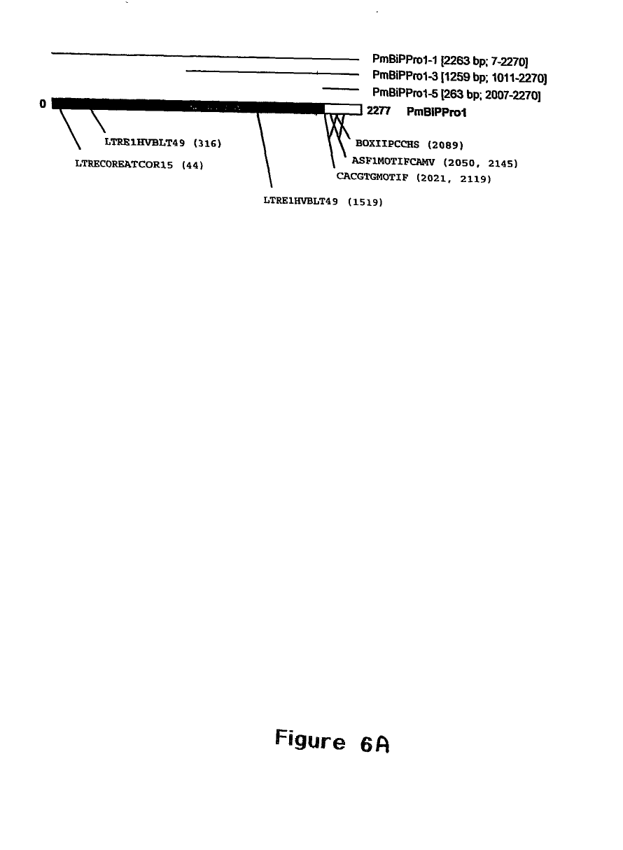

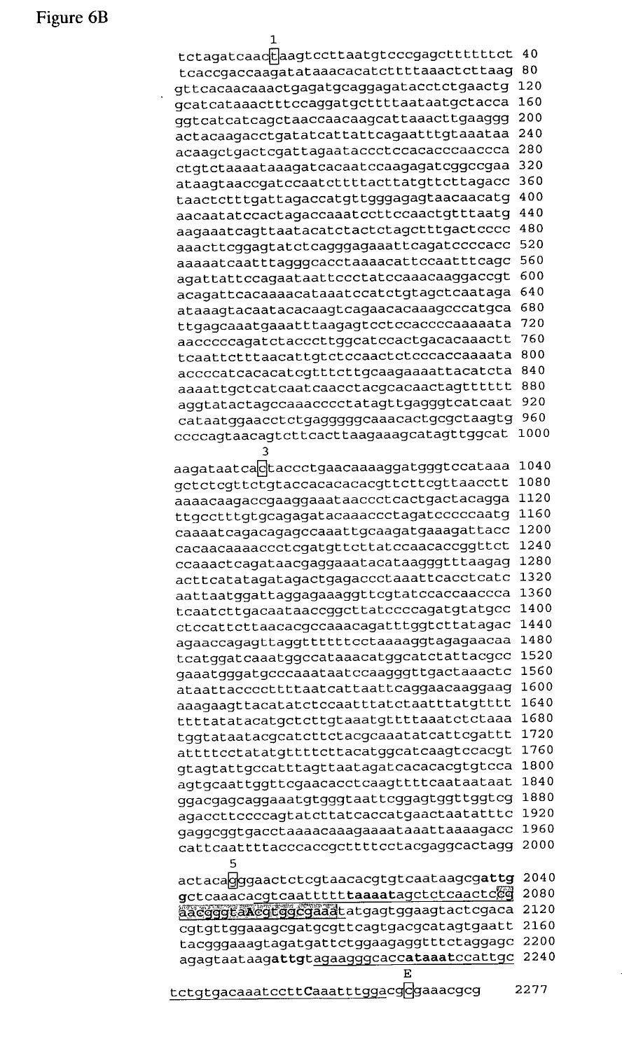

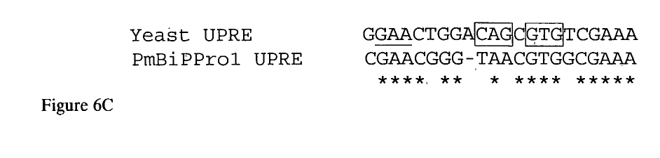

After a promoter has been identified, the promoter elements can be characterized, such as is described below for the PmBiP promoter (FIGS. 5A-G). This promoter contains a series of identifiable promoter elements. These elements appear in series in the genomic DNA as is shown schematically in FIG. 6. The space between the elements is referred to as “inter-element space.” An inter-element space can be modified through the addition, deletion, and/or substitution of nucleotides without loss of promoter activity.

A PmBiP promoter also can be modified by deleting elements from the promoter and/or duplicating elements within the promoter. One of ordinary skill in the art will appreciate that such modifications to the promoter can enhance promoter activity, inhibit promoter activity, or alter the level of tissue-specific expression of the promoter.

One of skill in the art will appreciate that, by modifying the order of the promoter elements, the number of the promoter elements, and/or the length of the inter-element space(s), one can modify promoter activity and/or native PmBiP promoter activity. However, in each case, the PmBiP promoter can drive expression of a gene operably linked to it. Assays for quantifying PmBiP activity as well as native PmBiP activity are provided below.

Protein: A biological molecule expressed by a gene and comprised of amino acids.

Purified: The term “purified” does not require absolute purity; rather, it is intended as a relative term. Thus, for example, a purified protein preparation is one in which the protein referred to is purer than the protein in its natural environment within a cell or within a production reaction chamber (as appropriate).

Recombinant: A “recombinant” nucleic acid is one having a sequence that does not occur naturally or having a sequence made by an artificial combination of two otherwise separated sequences.

This artificial combination can be accomplished by chemical synthesis or, more commonly, by the manipulation of isolated segments of nucleic acids, e.g., by genetic engineering techniques.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (e.g., human and mouse sequences), compared to species more distantly related (e.g., human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. To compare two nucleic acid sequences, the options can be set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq-i c:\seq1.txt-j c:\seq2.txt-p blastn-o c:\output.txt-q−1-r2.

A first nucleic acid is “substantially similar” to a second nucleic acid if, when optimally aligned (with appropriate nucleotide insertions or deletions) with the other nucleic acid (or its complementary strand), nucleotide-sequence identity occurs in at least about 60%, 75%, 80%, 85%, 90%, 92%, 95% or 98% of the nucleotide bases. (As used herein, “optimally aligned” sequences exhibit a maximal possible sequence identity). Sequence similarity can be determined by comparing the nucleotide sequences of two nucleic acids using BLAST as described above. Such comparisons may be made using the software set to default settings (expect=10, filter=default, descriptions=500 pairwise, alignments=500, alignment view=standard, gap existence cost=11, per residue existence=1, per residue gap cost=0.85).

One indication that two nucleic acid molecules are closely related is that the two molecules hybridize to each other under stringent conditions. Stringent conditions are sequence-dependent and are different under different environmental parameters. Nucleic acid molecules that hybridize under stringent conditions to a PmBiP cDNA and/or promoter sequence typically hybridize to a probe based on either an entire PmBiP gene or selected portions of the gene, respectively, under conditions described above.

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, 70%, 80%, 90%, 95%, 98%, or 99% sequence identity determined by this method.

An alternative (and not necessarily cumulative) indication that two nucleic acid sequences are substantially identical is that the polypeptide which the first nucleic acid encodes is immunologically cross reactive with the polypeptide encoded by the second nucleic acid.

To compare two amino acid sequences, the options of B12seq can be set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt j c:\seq2.txt-p blastp-o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (i.e., 1166−1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15÷20*100=75).

| | |
|---|---|
| | 1 20 |
| Target Sequence: | AGGTCGTGTACTGTCAGTCA |
| | \| \|\| \|\|\| \|\|\|\| \|\|\|\| \| |
| Identified Sequence: | ACGTGGTGAACTGCCAGTGA |

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least 75%, 80%, 85%, 90%, 95%, or 99% sequence identity.

When aligning short peptides (fewer than around 30 amino acids), the alignment can be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 60%, 70%, 75%, 80%, 85%, 90%, 95%, 98%, 99% sequence identity. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One of skill in the art will appreciate that these sequence identity ranges are provided for guidance only; it is possible that strongly significant homologs could be obtained that fall outside the ranges provided.

Transformed: A cell into which a nucleic acid molecule has been introduced, for example by molecular biology techniques. As used herein, the term transformation encompasses all techniques by which a nucleic acid molecule might be introduced into such a cell, including, but not limited to transfection with viral vectors, conjugation, transformation with plasmid vectors, and introduction of naked DNA by electroporation, lipofection, and particle gun acceleration.

Transgenic plant: A plant that contains recombinant genetic material (“transgene”) normally not found in a wild-type plant of the same species. Thus, a plant that is grown from a plant cell into which recombinant DNA is introduced by transformation is a transgenic plant, as are all offspring of that plant containing the introduced transgene (whether produced sexually or asexually).

Variants, fragments or fusion sequences: The production of proteins can be accomplished in a variety of ways. DNA sequences which encode for a protein (for example SEQ ID NO: 36) or fusion protein, or a fragment or variant of a protein, can be engineered to allow the protein to be expressed in eukaryotic cells, bacteria, insects, and/or plants. To obtain expression, the DNA sequence can be altered and operably linked to other regulatory sequences. The final product, which contains the regulatory sequences and the protein, is referred to as a vector. This vector can be introduced into eukaryotic, bacteria, insect, and/or plant cells. Once inside the cell the vector allows the protein to be produced.

A fusion sequence comprising a promoter, such as a PmBiP promoter, for example SEQ ID NOS: 31, 16, 17, or 18 (or variants, polymorphisms, mutants, or fragments thereof), linked to other amino acid sequences that do not inhibit the desired activity of the PmBiP promoter, for example the ability to promote gene expression.

One of ordinary skill in the art will appreciate that a DNA sequence, such as a PmBiP promoter, can be altered in numerous ways without affecting the biological activity of the promoter. For example, PCR can be used to produce variations in a PmBiP promoter DNA sequence. Such variants can be variants optimized for codon preference in a host cell used to express the protein, or other sequence changes that facilitate expression.

Vector: A nucleic acid molecule as introduced into a host cell, thereby producing a transformed host cell. A vector may include one or more nucleic acid sequences, such as an origin of replication, that permit the vector to replicate in a host cell. A vector also may include one or more selectable marker genes and other genetic elements known in the art.

Unless otherwise defined, all technical and scientific terms used herein have the same respective meanings as commonly understood by one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

A cDNA encoding the Douglas-fir luminal binding protein (PmBiP) was isolated by screening a Douglas-fir cDNA library with a previously isolated partial PmBiP cDNA clone. Four potential clones were isolated. The nucleotide and deduced amino acid sequence of the largest cDNA clone (PmBiP3) were used in subsequent experiments showing that PmBiP protein expression is developmentally and environmentally regulated. More specifically, PmBiP RNA levels to increased upon exposure to cold temperature, before and after fertilization and during germination.

The promoter responsible for the expression of the PmBiP protein was isolated by extracting genomic DNA from Douglas-fir spring-flush needles. The genomic DNA was digested with XbaI or SacI, re-circularized, subjected to PCR amplification, and identified via Southern blotting with a probe generated from a PmBiP cDNA clone.

Constructs containing the full-length PmBiP promoter (SEQ ID NO: 31) and deletions thereof (SEQ ID NOS: 16-18) were cloned upstream from the uidA (β-glucuronidase (GUS)) gene, and the activity of the promoter and fragments thereof determined. The PmBiP promoter (SEQ ID NO: 31) as well as deletions thereof (SEQ ID NOS: 16, 17, and 18) were highly active. In some cases, the PmBiP promoter and fragments thereof were more active than the commonly used 35S CaMV promoter.

### EXAMPLE 1

### Cloning of Douglas-fir PmBiP

Coastal Douglas-fir (Pseudotsuga menziesii [Mirb] Franco) seeds (seed-lot #952) were grown as previously described (Tranbarger et al., Gene 172:221-6, 1995). Germinating and young seedlings were collected at midday at the times indicated, frozen in liquid nitrogen, and stored at −80° C. until further use. Growth of young Douglas-fir seedlings (high elevation seed-lot #6485) used for seasonal expression analysis was as described in Ekramoddoullah et al. (Can. J For Res. 25:1137-1147, 1995). One needle from each of 112 trees was collected. The needles were pooled on the morning of the dates indicated, frozen in liquid nitrogen, freeze dried, ground to a powder, and stored at −20° C. until further use. Developing seeds were collected from an open-pollinated seed orchard during midday on the dates indicated at Pacific Forest Products Ltd., Saanichton, B.C., Canada. Developing seeds were promptly dissected from cones, frozen on dry ice, and stored at −80° C. until further use.

A partial length BiP cDNA clone from a Douglas-fir cDNA library prepared from poly A⁺ RNA isolated from 4-6-day old seedlings was used as a probe (Tranbarger et al., Gene 172:221-6, 1995). The cDNA was ³²P-labelled with a random primer DNA labeling kit (GIBCO BRL, Burlington, Ontario, Canada) and used to re-screen the cDNA library, according to the manufacturer's instructions (Strategene, La Jolla, Calif.), to obtain a full-length cDNA. Plasmid DNA from each positive clone was digested with EcoRI and electrophoresed on a 1% agarose gel. The DNA was transferred to a Zeta Probe™ membrane (BioRad, Mississauga, Ontario, Canada) for Southern blotting. Clones containing a BiP cDNA insert of an appropriate size were selected for DNA sequencing.

The largest cDNA clone was selected for double-stranded DNA sequencing using Sequenase™ (United States Biochemical, Cleveland, Ohio) and oligo primers synthesized on a PCR MATE™ 391 DNA synthesizer (Applied Biosystems, Mississauga, Ontario, Canada). Prediction of the signal sequence and signal-peptide cleavage site from deduced amino acid sequences was performed using the “SignalP” V1.1 World Wide Web Server (Nielsen et al., Protein Eng. 10:1-6, 1997). Amino-acid-sequence alignments were constructed using “CLUSTAL W” v1.7 (Thompson et al., Nucl. Acids Res. 22:4673-80, 1994). The phylogenetic tree was constructed using the PHYLIP package (Felsenstein, Cladistics 5:164-6, 1989). The amino acid sequences (and database accession numbers) used for this analysis were: Aspergillus awamorii (EMBL: Y12504), Aplysia californica (PIR: S24782), Arabidopsis thaliana 1 (DDBJ: D89341), Arabidopsis thaliana 2 (DDBJ: D89342), Caenorhabditis elegans (GENBANK: U56965), Drosophila melanogaster (PIR:JN0666), Echinococcus granulosus (GENBANK: M63605), Echinococcus multilocularis (GENBANK: M63604), Eimeria tenella (EMBL: Z66492), Gallus gallus (PIR: 150242), Glycine max A (GENBANK: U08384), Glycine max B (GENBANK: U08383), Homo sapiens (SWISS-PROT: P11021), Lycopersicon esculentum (SWISSPROT: P49118), Mesocricetus auratus (SWISS-PROT: P07823), Mus musculus (SWISS-PROT: P20029), Neurospora crassa (EMBL: Y09011), Nicotiana tabacum 4 (SWISS-PROT: Q03684), Nicotiana tabacum 5 (PIR: JQ1361), Oryza sativa (GENBANK: AF006825), Phytophthora cinnamomi (PIR: S38890), Plasmodium falciparum (EMBL: X69121), Phaeodactylum tricornutum (GENBANK: U29675), Rattus norvegicus (SWISS-PROT: P06761), Saccharomyces cerevisiae (SWISS-PROT: P16474), Spinacia oleracea (GENBANK: L23551), Trypanosoma brucei (GENBANK: L14477), Xenopus laevis (GENBANK:U62807), Zea mays E2 (GENBANK: U58208), and Zea mays E3 (GENBANK: U58209).

Douglas-fir genomic DNA was extracted from spring-flush needles by a modification of the “CTAB” method (De Vemo et al., Constructing Conifer Genomic Libraries: A Basic Guide, Information Report, Petawawa National Forestry Institute, Canadian Forest Service, PI-X-88, 1989). Aliquots of 10 μg of DNA were digested for 26 hours with restriction enzymes, then separated on a 0.7% agarose gel. Hybridization methods were based on those described in Lueders and Fewell, Biotechniques 16:66-7, 1994, as follows: The gel was incubated at room temperature with shaking in denaturing solution (0.5 N NaOH, 150 mM NaCl) for 30 minutes, rinsed in distilled water, and incubated in neutralizing solution (500 mM Tris-HCl pH 8, 150 mM NaCl) for 30 minutes. The gel was dried on a vacuum gel drier for 30 minutes with vacuum only, followed by 1 hour at 60° C. The dried gel (unblot) was probed with ³²P-labelled, random primed, PmBiP cDNA in hybridization solution (0.5 M Na₂HPO₄ pH 7.2, 7% SDS, 100 μg/ml denatured salmon sperm DNA) at 65° C. overnight, then washed at low stringency twice in hybridization solution for 45 minutes each at 65° C. The unblot was exposed for 7 days under a phosphor-imaging screen and developed using the STORM 820™ Phosphorimager (Molecular Dynamics, Sunnyvale, Calif.). Following development, the unblot was washed at high stringency twice in wash buffer (20 mM Na₂HPO₄ pH 7.2, 1% SDS) for 45 minutes each at 65° C. and exposed for 8 days and developed as above. Quantification was performed using the Image Quant NT™ software (Molecular Dynamics). Calculation of gene copy number was as described in Pasternak, Glick, and Thompson (eds.), Methods in Plant Molecular Biology and Biotechnology, CRC Press, Inc., Boca Raton, Fla., USA, pp. 29-36, 1993, using a Douglas-fir genome size of 25 pg per haploid nucleus (Ingle et al., Plant Physiol. 55:496-501, 1975).

### EXAMPLE 2

### PmBiP Antibodies

A synthetic peptide corresponding to the 13 C-terminal amino acids of the PmBiP deduced amino acid sequence (SEQ ID NO: 36) was synthesized at the University of Victoria Protein Micro-Chemistry Centre using a Model 430A peptide synthesizer (Applied Biosystems, Foster City, Calif.) with the “FastMoc” chemistry software. The peptide, with an additional Cys residue added to the N-terminal end, was then conjugated to a KLH carrier protein using the Imject™ kit and following the manufacturer's instructions (Pierce, Rockford, Ill.). The conjugated peptide was mixed with Freund's complete adjuvant and injected into New Zealand white rabbits. Subsequent booster injections were given at 2-week intervals using the conjugated peptide prepared in Freund's incomplete adjuvant. This antibody preparation was used as the primary antibody for Western blotting as described below.

### EXAMPLE 3

### Expression Pattern of Pm BiP mRNA and Protein

To determine the developmental regulation of PmBiP, the pattern of PmBiP mRNA and protein expression during seed and seedling development was examined using Northern and Western blotting. Western blotting also was used to determine whether seasonal variations exist in PmBiP protein levels in the needles of one-year-old seedlings.

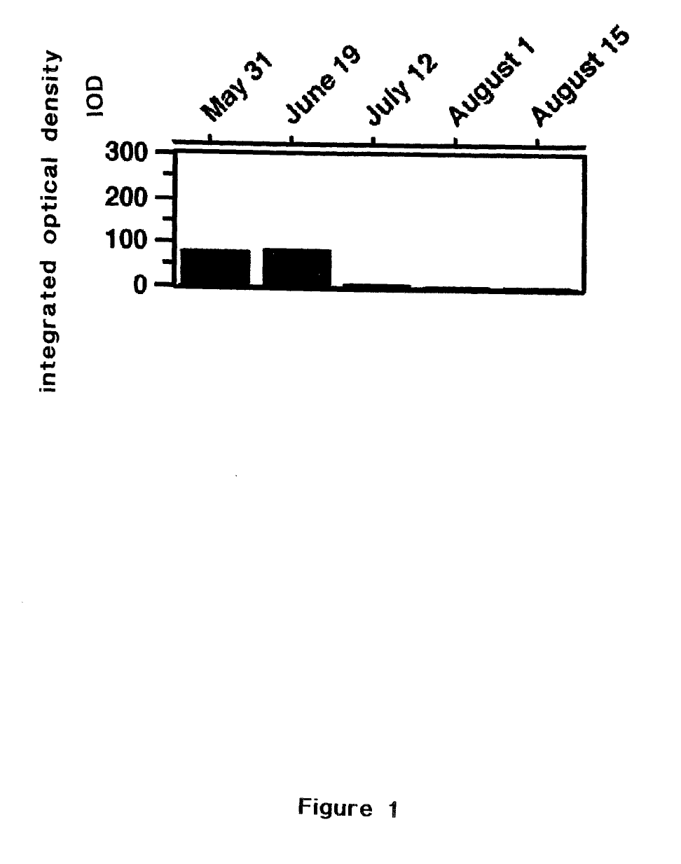

Total RNA was isolated from whole developing seeds collected at the time points shown in FIG. 1, as described in Kaukinen et al. (Plant Mol. Biol. 30:1115-28, 1996). The time points correspond to the following developmental stages, based on morphological characteristics of embryos established by Allen and Owens (The Life History of Douglas-fir, Environment Canada, Canadian Forestry Service, Ottawa, 1972): May 31—pre-fertilization, June 19—proembryo, July 12—early to mid-cotyledonary embryo, August 1—mid to late embryo, August 15—late to mature embryo.

The resulting RNA was separated on a 1% agarose/formaldehyde gel (20 μg per lane), and transferred to a Zeta-Probe GT membrane (BioRad, Mississauga, Ontario, Canada). Blots were probed with ³²P-labelled, random primed, PmBiP cDNA following the basic hybridization conditions described in the Zeta-Probe manual. Blots were stripped and re-probed with a PCR-amplified genomic fragment representing the Douglas-fir 18S rRNA gene to account for differences in the amount of RNA loaded per lane. Densitometry and adjustment for differences in the amount of RNA loaded per lane (calculation of integrated optical density) were performed as described in Tranbarger and Misra (Physiol. Plant 95:456-64, 1996). Densitometry was performed using the Chemilmager™ 4000 system (Alpha Innotech Corporation, San Leandro, Calif.).

As shown in FIG. 1, both before and soon after fertilization (May 31 and June 19, respectively), the amount of PmBiP mRNA observed in developing seeds was about 50- to 100-fold higher than the amount observed during embryogenesis (July 12-August 15). During embryogenesis, northern blotting of dissected material showed similar amounts of PmBiP mRNA in both the megagametophyte and developing embryo.

Western blot analysis was performed as follows. Protein extractions from whole developing seeds, mature seeds, germinating seeds, and young seedlings were performed by grinding approximately 100 mg of tissue in liquid nitrogen with a mortar and pestle. The powders were suspended individually in extraction buffer containing 65 mM Tris-HCl, pH 6.8, 1% SDS, 5% glycerol, and 2.5% β-mercaptoethanol, boiled for 5 minutes, frozen at −80° C. for 1 hour, boiled for 5 minutes, then centrifuged at 16,000×g for 25 minutes. The supernatants were collected and saved for further analysis.

For subcellular fractionations, approximately 5 g of tissue were frozen in liquid nitrogen and ground to a fine powder using a mortar and pestle. The individual powders were suspended and vortexed in buffer A (100 mM Tris-HCl pH 7.5, 250 mM sucrose, 2 mM MgCl₂, 10 mM KCl, 1 mM phenylmethylsulfonylflouride (PMSF), and 2.8 mM β-mercaptoethanol), then filtered through two layers of Miracloth™ (Calbiochem, La Jolla, Calif.). The filtrates were centrifuged at 25,000×g for 30 minutes. The supernatants were collected and centrifuged at 140,000×g for 1 hour. The supernatants (soluble fractions) were saved, and the respective pellets (microsomal fraction) were suspended in buffer B (50 mM phosphate buffer pH 7.5, 20% glycerol, and 10 mM β-mercaptoethanol). Microsomes were separated into soluble and membrane fractions according to Fujiki et al., J. Cell Biol. 93:97-102, 1982. To purify nuclei (nuclear fraction), the pellets from the 25,000×g centrifugation were resuspended in buffer A, layered on a 25%/75% Percoll™ (Sigma, Oakville, Ontario, Canada) step gradient, and centrifuged at 1000×g for 20 minutes. Nuclei were collected from the 25%/75% interface, washed 2× in buffer A, and suspended in buffer B.

Protein concentrations were determined by the BioRad Reagent protein assay (BioRad). Extraction and quantification of needle proteins from seasonal samples and densitometry of western blots were performed as described in Ekramoddoullah et al. (Can. J. For. Res. 25:1137-47, 1995). Protein samples were suspended in protein sample buffer (12.5 mM Tris-HCl pH 6.8, 2% SDS, 10% glycerol, 5% β-mercaptoethanol, and 0.1% bromophenol blue), boiled for 3 minutes, and separated by SDS-PAGE using the Mini-PROTEAN II™ gel electrophoresis system (BioRad) with a 4% (w/v) acrylamide stacking gel (80 volts; constant voltage) and an 11% (w/v) acrylamide separating gel (200 volts; constant voltage). The proteins were stained with Coomassie brilliant blue R250 or transferred to nitrocellulose membranes (Schleicher & Schuell, Keene, NH) using a Mini-Trans-Blot™ cell (BioRad) at 100 volts for 1 hour in transfer buffer (25 mM Tris, 190 mM glycine, 20% methanol, and 0.1% SDS). The membranes were blocked overnight at 4° C. in Tris-buffered saline (“TBS”; 20 mM Tris, 500 mM NaCl; pH 7.5) containing 0.05% Tween-20 (“TTBS”), incubated with primary antibody (diluted 1:3000 in TTBS) for 90 minutes at room temperature, then washed two times with TTBS (5 minutes each). The membranes were then incubated with an alkaline phosphatase-conjugated goat anti-rabbit antibody (1:3000 dilution in TTBS) (Cedar Lane Laboratories Ltd., Homby, Ontario, Canada) for 45 minutes at room temperature, followed by washing in TTBS (5 minutes) and TBS (5 minutes). Immunoreactive bands were visualized by incubating the membrane with 5-bromo-4-chloro-3-indolyl-phosphate (0.165 mg/ml) and nitroblue tetrazolium (0.33 mg/ml) as substrate in buffer containing 100 mM NaHCO₃ pH 9.8 and 1 mM MgCl₂.

Western blot analysis of seeds collected at various stages of seed development demonstrated that the amount of PmBiP protein also was high before and soon after fertilization, and decreased thereafter.

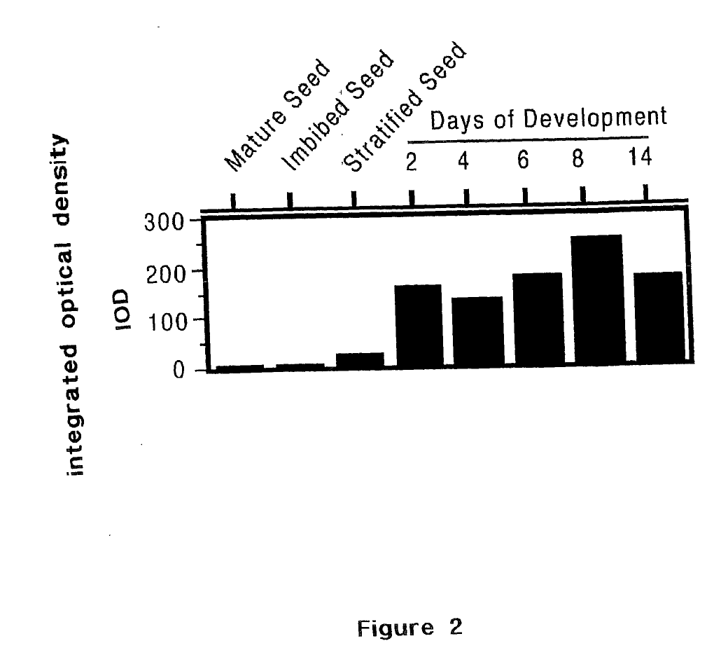

To measure expression of PmBiP RNA during germination and early seedling development, total RNA was isolated from tissue collected at the indicated time points shown in FIG. 2 and subjected to northern blot analysis (20 μg per lane) as described above using the PmBiP cDNA as probe. As shown in FIG. 2, following imbibition and stratification, PmBiP mRNA increased slightly. Upon exposure of the seeds to germination conditions, the amount of PmBiP mRNA increased to levels greater than the level observed during early stages of seed development. Levels increased 150- to 200-fold over levels observed in mature or imbibed seeds after only a 2-day exposure to germination conditions. PmBiP mRNA amounts were highest after 8 days, approximately 250-fold higher than observed in mature seeds. The amount of PmBiP protein did not show an increase until 8 days after exposure of the stratified seeds to germination conditions, with the highest amounts appearing after 14 days.

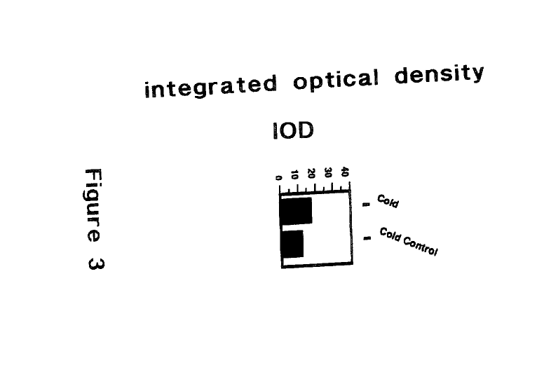

Temperature regulation of PmBiP protein expression was observed at both the mRNA level and the protein level. 14-day-old seedlings were subjected to cold treatment, and mRNA and protein levels were assessed using northern and western blotting. After the cold treatment both protein levels and mRNA levels (FIG. 3) increased.

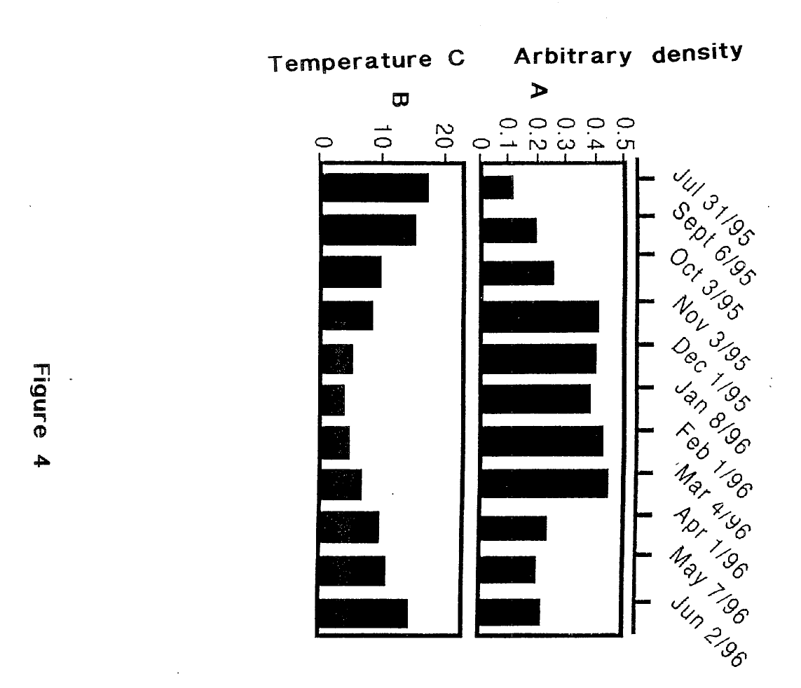

The abundance of PmBiP protein was followed over a one-year period in needles collected from 1-year-old Douglas-fir seedlings kept under natural day-length and temperature in an outdoor shelter house. Total protein was isolated from the needles at the indicated times and subjected to western blot analysis as described above (15 μg per lane). Following blot development, immunoreactive bands were quantified using scanning densitometry and displayed graphically in units of arbitrary density. As shown in FIGS. 4A and 4B, PmBiP protein levels showed seasonal variation, with the highest amounts occurring in needles taken from seedlings during the winter, when the monthly average temperature was below 10° C.

### EXAMPLE 4

### Characterization of the PmBiP Promoter

Inverse PCR and Cloning

Inverse PCR was conducted based on the method described in Ochman et al., Amplification of Flanking Sequences by Inverse PCR, 1980, and Innis et al. (eds.) PCR Protocols; A Guide to Methods and Applications, Academic Press Inc., San Diego, 1990, as follows. Douglas-fir genomic DNA was extracted from spring-flush needles by a modification of the CTAB method of De Verno et al., Constructing Conifer Genomic Libraries; A Basic Guide, Petawawa National Forestry Institute, Canadian Forest Service, 1989. Approximately 18 μg of DNA was digested with XbaI or SacI overnight at 37° C. Each reaction was heat-inactivated at 65° C. for 20 minutes, then suspended in 10 ml of ligation buffer (50 mM Tris-HCl pH 7.4, 10 mM MgCl₂, 10 mM dithiothreitol, and 1 mM ATP) with 0.02 Weiss units/μL T4 DNA ligase for 16 hours at 15° C. Circularized DNA was precipitated by the addition of 1/10 volume of 2.5 M ammonium acetate, followed by 2 volumes of −20° C. 100% ethanol on ice, then centrifuged at 25,000×g for 10 minutes at 4° C. Precipitated DNA was suspended in 60 μL of sterile distilled H₂O of which 5 μL was subjected to PCR using Taq PCR MasterMix™ (QIAGEN, Mississauga, Ontario, Canada) and 250 pmol of the following primers in a 100-μL reaction: XbaI used primer combinations p5-3z8 (5′-AAT GAA AGC GAA GTG ACA CC-3′; SEQ ID NO: 19) and p14-5a4 (5′-CAG AAC CAT TAA CAA GAG CAA GAT 3′; SEQ ID NO: 20) or p14-5z1.1 (5′-AAC CAG CAG TGA TAA ACG CC-3′; SEQ ID NO: 21) and p14-5a4; SacI used primer combinations p5-3z8 and p14-5a3 (5′-TAT GGT TTG GAT AAA AAG GGA G-3′; SEQ ID NO: 22) or p14 5z1.1 and 14-5a3. Conditions for PCR consisted of 1 cycle of denaturing at 95° C. for 5 minutes and 1 minute at 75° C., 30 cycles of denaturing at 94° C. for 1 minute, primer annealing at 56° C. for 1 minute and an extension of 72° C. for 2 minutes, followed by a final elongation step at 72° C. for 5 minutes. Aliquots of each reaction (20 μL) were separated on an agarose gel and subjected to Southern blotting to identify potential promoter fragments. PCR reactions containing positive fragments were cloned into the pCR®2.1-TOPO vector using the TOPO TA™ Cloning Kit (Invitrogen, Carlsbad, Calif., U.S.A.) according to the manufacturer's instructions. Colonies containing an appropriately sized insert were screened using PCR with the appropriate primers followed by Southern blotting. Plasmid DNA for colony screening using PCR was obtained by suspending colonies in 200 μL of distilled H₂O, followed by incubation at 85° C. for 5 minutes. Samples were centrifuged at 16,000×g for 5 minutes, and 36 μL were removed and used as a template for PCR.

DNA Sequencing

PmBiP promoter (SEQ ID NO: 31) and expression constructs were sequenced using the Big Dye™ Superscript Terminator Cycle sequencing Ready Reaction (Perkin Elmer) and oligo primers with the ABI Prism automated 377 DNA Sequencer (Perkin Elmer). Plasmid DNA for sequencing was isolated using the Wizard™ 373 DNA Purification System (Promega, Madison, Wis.). DNA sequence trace files were assembled using the DNASTAR™ program SeqMan (DNASTAR Inc, Madison, Wis.).

Analysis of the PmBiPPro1 DNA sequence and identification of putative regulatory elements were performed done by searching the plant cis-acting DNA regulatory database (PLACE; Higo et al., Nucl. Acids Res. 27:297-300, 1999).

Southern Blotting

DNA was electrophoresed on a 1% agarose gel and transferred to a Zeta Probe™ membrane (BioRad, Mississauga, Ontario, Canada) according to the manufacturer's instructions. The PmBiP3 cDNA was ³²P-labelled with a random-primers DNA labeling kit (GIBCO BRL, Burlington, Ontario, Canada). Hybridization and washing were performed according to the standard protocol in the Zeta Probe manufacturer's instructions. Blots were exposed using Kodak X-OMAT AR film (Eastman Kodak Company, Rochester, N.Y.) overnight at −80° C.

The resulting inverse PCR product was 2760 bp, and contained 2277 bp of sequence referred to as PmBiPPro1 (SEQ ID NO: 31) immediately upstream of the PmBiP3 cDNA sequence. As shown in FIGS. 5A-G and FIG. 6B, PmBiPPro1 (SEQ ID NO. 31) includes several possible cis-acting elements. Predicted promoter regions (−40 to +10) using the Neural Network Promoter Prediction program (Reese and Eeckman, 1995 Novel Neural Network Algorithms for Improved Eukaryotic Promoter Site Recognition. In The Seventh International Genome Sequencing and Analysis Conference (Hilton Head Island, S.C.); Reese et al., 1996. Large Scale Sequencing Specific Neural Networks for Promoter and Splice Site Recognition. In Biocomputing: Proceedings of the 1996 Pacific Symposium, L. Hunter and T. E. Klein, eds. (Singapore: World Scientific Publishing Co)) are underlined with the putative transcriptional start site indicated in bold capital.

A putative UPRE was identified (nucleotides 2079-2099 of SEQ ID NO: 31). An alignment of a yeast UPRE to the PmBiPPro1 UPRE, indicates that certain resicudes in the UPRE are more critical for promoter function than others (FIG. 6C). For example. Possible TATA-boxes identified using the Hamming-clustering method are indicated in bold (Milanesi et al, Computer Applications in the Biological Sciences, 12:399-404, 1996). The nearest upstream CAAT boxes to each TATA-box are also indicated in bold. The first residue of PmBiPPro1-1 (1) (SEQ ID NO: 16), PmBiPPro1-3 (3) (SEQ ID NO: 17), and PmBiPPro1-5 (5) (SEQ ID NO: 18) promoter reporter construct is boxed as is the 3′ end of each construct (E).

Several of the identified cis-acting elements may be responsible for promoter activity in response to environmental changes (i.e., inducible), such as light, temperature, wounding, and water stress. Other identified promoter elements indicate that the endogenous PmBiP promoter (SEQ ID NO: 31) contains negative regulatory regions. One of skill in the art will appreciate that the identification of these regions, and the other elements shown in FIGS. 5A-G and 6A-C, facilitates the subsequent modification of the PmBiP promoter (SEQ ID NO: 31). Thus, the PmBiP promoter (SEQ ID NO: 31) can be modified via deletion of negative regulatory elements to increase transcription, or to alter the induciblity of the promoter via the addition or deletion of inducible elements.

### EXAMPLE 5

### PmBiP Promoter Variants

This example describes experiments in which the full-length PmBiP promoter was shortened, and the activity of these truncated promoters tested. The isolation and sequencing of the PmBiP promoter (SEQ ID NO: 31) facilitated the creation of the deletion constructs PmBiPPro1-1, PmBiPPro1-3, and PmBiPPro1-5 (FIG. 6A). These deletion constructs were used to stably transform Arabidopsis, potato, and tobacco, and to transiently transform Douglas-fir zygotic embryos. Similar experiments can be performed on other variant PmBiP promoter sequences and in other organisms, such as other plants.

Construction of Vectors Containing PmBiP Promoter Sequences

The following promoter-gene fusions were constructed for expression in the cytosol. Plasmids were constructed from parent plasmids pBI121 and pBI221 for stable and transient expressions, respectively. PmBiP promoter constructs were generated using PCR with either Taq polymerase (PmBiPpro1-1; Quiagen) or DeepVent™ polymerase (PmBippro1-3 and PmBiPpro1 5; NEB) and the PmBiPpro1 clone as template. The primers, containing HindIII and XbaI sites, used for amplification of the various promoter constructs, employed the same 3′-primer (5′-TCG AAG CGC AAA TCT AGA GTT TAA ACT TCC-3′; SEQ ID NO: 23) and the following 5′-primers: PmBiPPro1-1 (5′-AAG AAG GCA AGC TTT CAA CTA A-3′; SEQ ID NO: 24), PmBiPPro1-3 (5′-GCA TAA GAA AGC TTC TAC CCT G-3′; SEQ ID NO: 25), and PmBiPPro1-5 (5′-GCA CTA GGA AGC TTG GGA ACT C-3′; SEQ ID NO: 26).

Following restriction digestion, the resulting products (PmBiPpro1-1, 2263 bp, SEQ ID NO: 16; PmBiPpro1-3, 1259 bp, SEQ ID NO: 17; PmBiPpro1-5, 263 bp, SEQ ID NO: 18) were cloned into the HindIII and XhaI sites of pBI221, replacing the CaMV 35S promoter (˜0.8 kb). The resulting plasmids, containing PmBiP promoter sequences, were labeled pPRO1-1221, pPRO1-3221, and pPRO1-5221. Replacing the HindIII-XbaI fragment in pBI121 (containing the CaMV 35S promoter) by HindIII-XbaI fragments from pPRO1-1221, pPRO1-3221, and pPRO1-5221 respectively, created the plasmids pPRO1-1121, pPRO1-3121, and pPRO1-5121.

Transient Expression

Growth of Douglas-fir embryos used for transient expression was as follows. Interior Douglas-fir (Pseudotsuga menziesii [Mirb.] Franco) seeds (seed-lot 8912) were imbibed for 2 days at 4° C., then surface-sterilized in 50% industrial bleach (6% sodium hypochlorite) for 20 minutes at room temperature. Embryos were aseptically dissected from seeds and placed on woody plant medium (WPM; Table 1; Lloyd and McCown, Proc. Int. Plant Prop. Soc. 30:421-7, 1980) at 22° C. in the dark for 16 hours before particle bombardment.

Germinated Douglas-fir zygotic embryos were bombarded using the model PDS-1000/He Biolistic® Particle Delivery System (BioRad). DNA (pBI221 plasmid derivatives) was coated onto gold particles (1-3 μm diameter; Sigma-Aldrich Canada Ltd, Oakville, Ontario, Canada) as described by Jefferson, Plant Mol. Biol. Rep. 5:387-405, 1987, as follows. A gold suspension (60 mg/ml) was prepared in 50% glycerol of which 15 μL was placed in 1.5-ml microfuge tubes with 4.2×10¹¹ copies of either a CaMV 35S:GUS plasmid (pBI221, 5700 bp; Clontech Laboratories Inc, Palo Alto, Calif.), a plasmid containing PmBiPpro1-1 (SEQ ID NO: 16) (7188 bp), a plasmid containing PmBiPpro1-3 (SEQ ID NO: 17) (6184 bp), or a plasmid containing PmBiPpro1-5 (SEQ ID NO: 18) (5189 bp), 15 μL of 2.5 M CaCl₂, and 6 μL of 0.1 M spermidine with continuous vortexing.

The particles were allowed to settle on ice, then pelleted by a brief centrifugation. The supernatant was discarded, and 70 μL cold 70% ethanol was added without disturbing the pellet. The 70% ethanol was removed, an additional 70 μL cold 100% ethanol was added and removed without disturbing the pellet and the resulting particles suspended in 30 μL cold 100% ethanol with slow vortexing. Aliquots (10 μl) were placed on a macrocarrier disk and allowed to dry in the presence of silica gel desiccant.

Each bombardment delivered 1.4×10¹¹ constructs and was conducted using the following parameters. The gap distance between the rupture disk and macrocarrier was 0.6 cm, the macrocarrier travel distance was 0.6 cm, the target tissue distance was 8 cm from the microcarrier launch assembly platform, the sample chamber vacuum was 25 inches of mercury, and rupture pressure was 1550 psi. Tissue then was incubated on WPM at 22° C. in the dark for 48 hours prior to histochemical GUS staining. Following GUS staining (see below), the number of blue spots were counted under a stereo dissecting microscope.

Arabidopsis Transformation

Approximately 10-20 A. thaliana (L.) Heynh. seeds of ecotype Columbia were placed on a nylon screen covering moistened Sunshine™ mix #3 soil (Sun Gro Horticulture, Bellevue, Wash.) on 10-cm-diameter pots, and then covered with Saran Wrap™ secured with an elastic band. Pots were then placed at 4° C. for 2 days to promote uniform germination. Pots were placed in a growth chamber with an 18 hours 24° C. day/6 hours 22° C. night cycle with 150 μEm⁻²s⁻¹ of light. The Saran Wrap™ was removed when plants began to push against its surface (approximately 2 days).

A. thaliana plants were transformed according to the method of Clough and Bent (Plant J. 16: 735-43, 1998). Agrobacterium tumefaciens strain MP90 carrying the plasmid CaMV 35S:GUS or PmBiPPro1 (SEQ ID NO: 16):GUS was grown to stationary phase in liquid culture at 28° C., 250 rpm, in sterile LB broth (10 g tryptone, 5 g yeast extract, 5 g NaCl per liter of water) containing 50 μg/ml kanamycin and 10 μg/ml gentamycin. Cells were harvested by centrifugation for 20 minutes at room temperature at 5500×g, then suspended in infiltration medium (5.0% sucrose and 0.05% Silwet L-77 (Lehle Seeds, Round Rock, Tex.) to a final OD₆₀₀ of approximately 0.8 prior to use. The above-ground portions of plants were dipped in infiltration medium containing Agrobacterium for 3-5 sec with gentle swirling 2 days after removal of the primary bolt. One subsequent dip was made 7 days later. Following each dip, the plants were covered with a plastic bag for 24 hours to retain moisture. Plants were grown normally and fed with HI-SOL™ 18-24-12 soluble plant food (Ig/L; Green Valley Fertilizer, Abbotsford, B.C, Canada) once a week via sub-irrigation. Plants were no longer watered after seed pods began to turn brown. When plants were fully dried, they were placed in a brown paper bag for 1 week prior to collecting seed. Seeds were collected by manually rubbing plants and pods, then filtering the debris through a 0.707-mm mesh sieve (W. S. Tyler Company of Canada Ltd., St. Catherines, Ont., Canada) several times until the seeds were reasonably free of other matter.

Seeds were sterilized using vapor-phase sterilization as follows (Clough and Bent, Plant J. 16:735-43, 1998). Collected seeds were placed in 15-ml conical tubes (2-3 ml seeds per tube) with lids attached loosely. Tubes were placed in a rack inside a plastic vacuum dessicator (Bel-Art #42025, 240-mm internal diameter) containing a 250-ml glass beaker with 150 ml bleach (5.25% sodium hypochlorite). Concentrated HCl (5 ml) was placed in a 10-ml glass beaker and floated on top of the bleach solution. The lid was placed on the dessicator and a slight vacuum applied. The dessicator was shaken slightly to spill the concentrated HCl into the bleach to liberate chlorine gas for overnight sterilization. Sterile seeds were sprinkled on 150×15 mm² selection plates (½ MS media/0.8% agar/1% sucrose, 50 μg/ml kanamycin, 100 μg/ml ampicillin) and placed in dark at 4° C. for 2 days. Plates were removed and placed in a growth chamber with 16 hours/8-hours light/dark at 22° C. for 2 weeks. Healthy green transformants were selected and placed in moist soil in a growth chamber with an 18-hour 24° C. day/6-hour 22° C. night cycle with 150 μEm⁻²s⁻¹ of light. The plants were covered with Saran Wrap™ for the first 2 days.

Tobacco and Potato Transformation

Tobacco plants (Xanthi) and 4-6 week old potato plants (Desiree) were grown in Majenta jars on hormone free MS medium (Murashige and Skoog, Physiol. Plant 115:473-97, 1962) under a 16 hours light/8 hours dark photoperiod at a constant temperature of 23° C.

Leaf strips from tobacco and stem segments (5-10 mm pieces) and leaves (cut at the base) from potato were pre-cultured upside down for 3-5 days on MS 104 medium (MS medium supplemented with 1 μg/ml BAP, 0.1 μg/ml NAA, pH=5.7). Explants were incubated in S2 medium (MS medium without agar but supplemented with 0.5 g/L MES and 20 g/L mannitol) inoculated with a 1:200 (v:v) dilution of an overnight culture of Agrobacterium tumefaciens strain MP90 for 2-3 days under low light intensity. An overnight culture of Agrobacterium tumefaciens was grown at 28° C. in LB media supplemented with 50 μg/ml kanamycin and 10 μg/ml gentamycin. Explants were incubated at low light intensity on Stage I medium (MS medium supplemented with 6 g/L agarose (instead of 8 g/L agar), 200 mg/L glutamine, 600 mg/L MES, 500 mg/L PVP, 20 g/L mannitol, 20 g/L glucose, 40 mg/L adenine-SO₄, 2.5 mg/L zeatine-riboside, 0.1 mg/L NAA, and 0.02 mg/L GA₃) for 3-5 days followed by transfer to Stage II medium (Stage I medium supplemented with 100 μg/ml kanamycin and 500 μg/ml cefotaxime) for 7-12 days to initiate growth of callus. To initiate growth of shoots, explants containing callus were transferred to Stage III medium (Stage II medium containing no NAA) for 5-7 weeks. Following shoot formation, plantlets were transferred to rooting medium (MS medium supplemented with 50 μg/ml kanamycin and 250 μg/ml cefotaxim) and grown as described for parent plants.

Histochemical GUS Staining

GUS staining was performed as described by Jefferson (Plant Mol. Biol. Rep. 5:387-405, 1987). Briefly, tissue from particle bombardment or transgenic plants was immersed in solution containing 1 mM X-Gluc, 100 mM sodium phosphate buffer pH 7.0, 10 mM EDTA, 0.5 mM potassium ferricyanide, 0.5 mM potassium ferrocyanide, 0.1% triton X-100, and incubated overnight at 37° C.

In vitro GUS assay

Fresh plant tissue was placed in a 1.5-ml Eppendorf tube containing ice-cold lysis buffer (50 mM sodium phosphate pH 7.0, 10 mM EDTA, 0.1% Triton X-100, 0.1% sarkosyl, 10 mM β-mercaptoethanol, and 0.02 g/ml insoluble polyvinylpyrrolidone (PVP)), and homogenized using a glass pestle connected to a Baruant series 10 mixer (Barnant Company, Barrington, Ill.). Homogenates were centrifuged at 16,000×g for 15 minutes at 4° C. Supernatants were collected and assayed for protein using the method of Bradford (Anal. Biochem. 72:248-54, 1976). GUS activity was measured in 100 μL extraction buffer (without PVP) containing 6 μg of total protein and 1 mM p-nitrophenyl-β-D-glucuronide as substrate at 37° C. using a Thermomax™ microplate reader and Softmax™ Pro v3.1 software (Molecular Dynamics Corporation). Absorbance was measured at 405 nm every 5 minutes or after 18 hours.

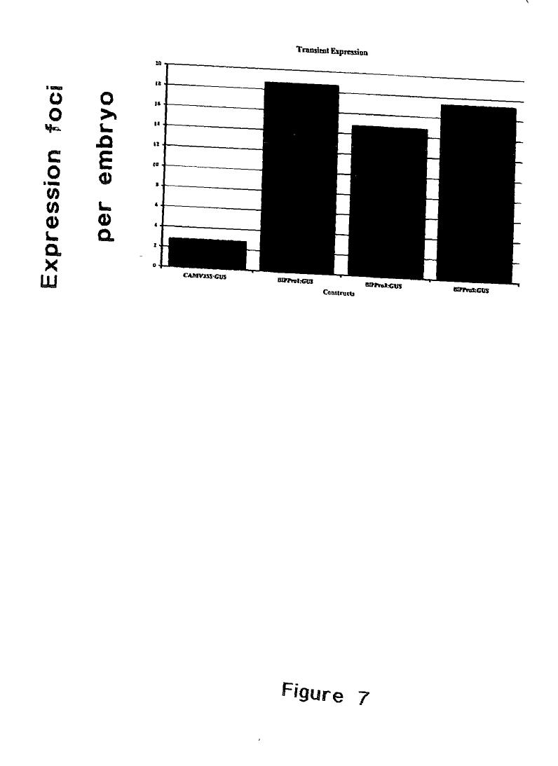

Douglas-fir zygotic embryos were transiently transformed with construct containing the GUS open reading frame under the control of PmBiPpro1-1 (SEQ ID NO: 16), PmBiPpro1-3 (SEQ ID NO: 17), PmBiPpro1-5 (SEQ ID NO: 18), or CaMV35S. As shown in FIG. 7, all PmBiP promoter constructs were capable driving the expression of GUS. Even the relatively weak PmBiP promoter construct (PmBiPpro1-3) drove expression of the transgene at a rate 7-fold higher than the rate exhibited by the control CaMV35S promoter construct (average expression levels taken over two trials, ten embyros per trial; data reported as an average number of expression foci per embryo).

Potato and tobacco plantlets stably transformed with either the GUS open reading frame under the control of PmBiPpro1-1 (SEQ ID NO: 16), PmBiPpro1-3 (SEQ ID NO: 17), PmBiPpro1-5 (SEQ ID NO: 18), or CaMV35S showed that even PmBiPpro1-5 (SEQ ID NO: 18), the shortest fragment, was capable of driving GUS expression. Moreover, the PmBiPpro1-5 construct (SEQ ID NO: 18) showed comparable promoter activity to that of the control CaMV35S construct.

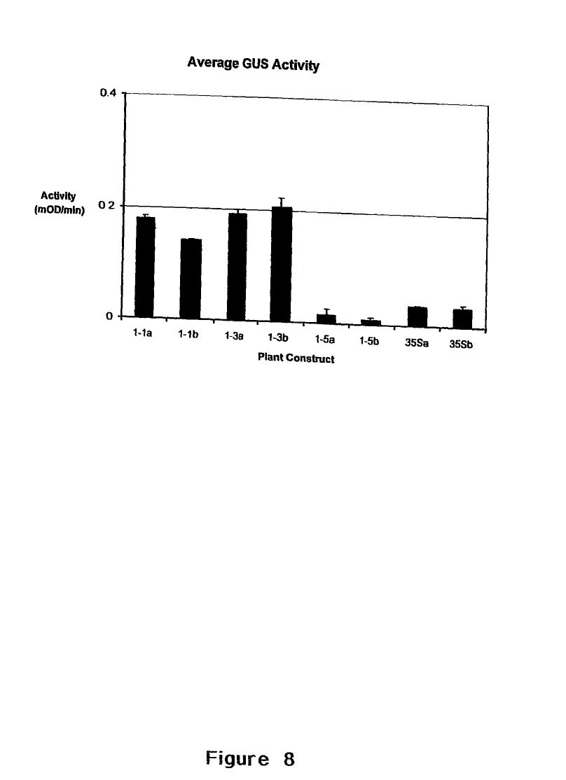

Stably transformed 19-day-old Arabidopsis plants had the highest level of GUS expression in plants transformed with the PmBiPpro1-3 construct. However, plants transformed with PmBiPpro1-1 (SEQ ID NO: 16) showed very similar levels of expression (FIG. 8; results represent the average and standard deviation of three trials for each plant extract).

### EXAMPLE 6

### Inducement of PmBiP Promoter by Wounding

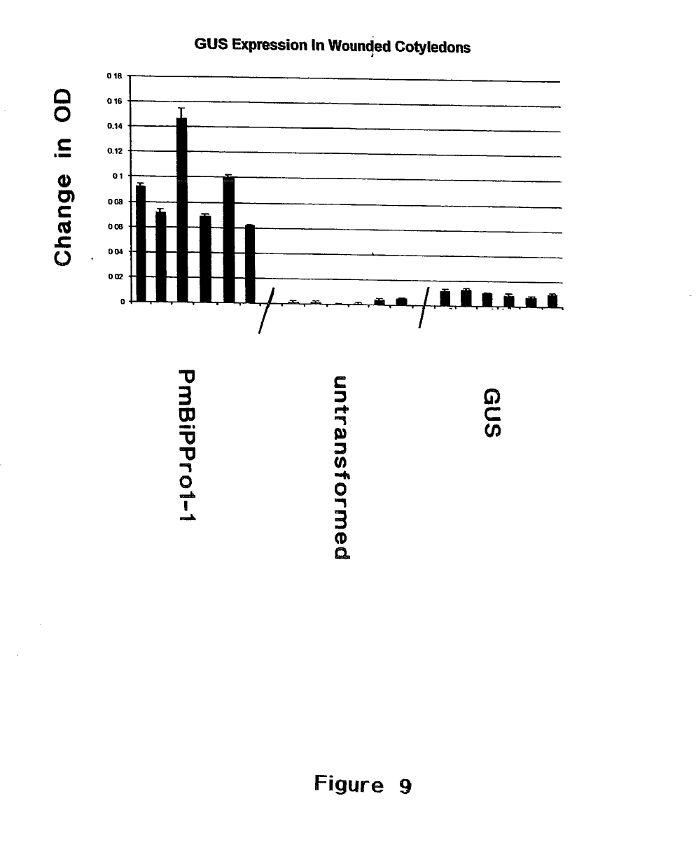

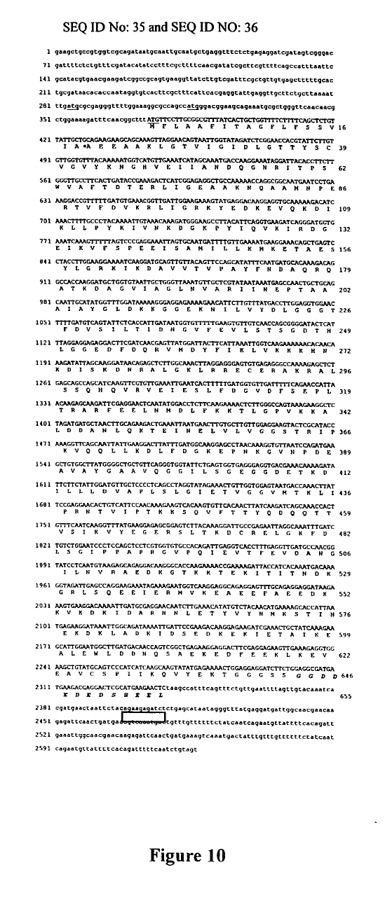

The ability of the PmBiP promoter to be induced via wounding was tested in stably transformed 21-day-old Arabidopsis cotyledons (FIG. 9). One cotyledon was wounded by pinching with forceps, and the second cotyledon was used as a control. GUS activity was measured in 6 μg of total protein extracted, 18 hours after wounding as described above. GUS activity is measured as change in absorbance (AOD) after 18 hours. GUS assays also were performed on wounded untransformed and CaMV35S:GUS plant cotyledons. Results represent the average and standard deviation of three trials for each plant extract induced via wounding. The results demonstrate that PmBiPpro1-1 (SEQ ID NO: 16) is wound-inducible.

This data, coupled with the PmBiP protein expression data described above, demonstrate that the PmBiP promoter is inducible at least by wounding and upregulated by temperature alterations. These attributes make this promoter particularly useful in situations where it is desirable to produce a protein at cold temperatures (i.e., where increased protein stability is desired). “Cold” implies that the plant is being grown at a colder temperature than otherwise would be optimal for host growth. For example, a plant or a plant part (i.e., a leaf, or stem) can be wounded and placed in a cold temperature, such as at less than 20° C., less than 15° C., or less than 10° C. Additionally, the table of cis-acting elements provided in FIGS. 5A-5G and 6A-C demonstrate that the PmBiP promoter may be inducible by other environmental factors.

### EXAMPLE 7

### Modifications of the Douglas-fir PmBiP Promoter

The structure of a promoter determines the level of mRNA expression as well as specificity of the promoter. However, expression levels and/or specificity can be maintained when deletions, substitutions, and/or additions are made to the promoter sequence. Hence, the scope of the disclosure encompasses PmBiP promoters that have been modified through the incorporation of deletions, substitutions, and/or additions. However, variant PmBiP promoter sequences will continue to exhibit promoter activity, or native PmBiP promoter activity, as described above.

One method of modifying a PmBiP promoter is by inserting additional promoter elements into the promoter sequence. For example, the promoter can be modified such that an E-box motif, RY-repeated element, AT-rich region, ACGT-core element, opaque-2-like binding site, a UPRE element, and/or a conserved gymnosperm-like region is added. One of skill in the art will appreciate that standard molecular biology techniques can be used to insert one or more of these elements into a promoter sequence. The modified promoter then can be transiently transfected into gymnosperm, monocot, or dicot tissue and the tissue can be tested for transgene expression as described in the above examples.

Similarly, one or more of the existing promoter elements can be deleted from a PmBiP promoter sequence. The modified promoter can be tested for transcriptional activity and specificity. Given the disclosure of PmBiP promoters and the above-described materials and methods, it also is possible to make additions and deletions and test for promoter activity as described in the above examples.

The PmBiP promoter also can be modified such that the inter-element spaces contain deletions, insertions, and/or substitutions. One of ordinary skill in the art can use standard molecular biology techniques to insert additional nucleic acid residues into the inter-element spaces, delete nucleic acid residues from the inter-element spaces, and/or substitute other sequences into the inter-element spaces. However, regardless of the number and combination of insertions, deletions, and substitutions, the PmBiP promoter will maintain promoter activity. In some cases, the promoter will maintain native PmBiP promoter activity.

Cloning Nucleic Acid Sequences Encoding PmBiP

Provided with the nucleic acid sequence of the Douglas-fir PmBiP promoter (SEQ ID NO: 31), one of ordinary skill in the art will appreciate that several different methods can be used to isolate the Douglas-fir PmBiP promoter (SEQ ID NO: 31). One example of such a method is the polymerase chain reaction (PCR) (U.S. Pat. No. 4,683,202 to Mullis; and Saiki et al., Science 239:487-91, 1988).

After isolation, the PmBiP promoter sequence is useful for driving the expression of transgenes.

When using PCR to isolate a sequence encoding a gene, a first primer can be designed that targets the extreme 5′-end of the sequence, and a second primer can be designed that targets the extreme 3′-end of the sequence. These primers can be used to generate multiple copies of the promoter sequence. The copies are isolated by separation on an agarose gel. The fragment of interest is then removed from the gel and ligated into an appropriate vector.

Alternatively, a promoter can be created by engineering synthetic strands of DNA that partially overlap each other (Beaucage and Caruthers, Tetrahedron Letters 22:1859-69, 1981; Matthes et al., EMBO J. 3:801-5, 1984). The synthetic strands are annealed, and a DNA polymerase fills in the single-stranded regions. The resulting synthetic double-stranded DNA molecule can be cloned into a vector.

For use as primers and probes, nucleic acid sequences can contain at least 15 contiguous nucleic acid molecules of the sequences shown in SEQ ID NOS: 16, 17, 18, and 31, or the complementary strand of such sequences. The nucleic acid sequences are useful for performing hybridization protocols, such as northern blots or Southern blots as described in Sambrook et al. (eds.), Molecular Cloning, A Laboratory Manual, 2d ed., vol. 1-3, Cold Spring Harbor Laboratory Press, Cold Spring Harbor, N.Y., 1989.

These hybridization protocols can be used to identify nucleic acid sequences that are substantially similar to the sequences shown in SEQ ID NOS: 16, 17, 18, and 31. A successful hybridization to such sequences indicates that the analogous nucleic acid sequence hybridizes to the oligonucleotide probe that comprises at least a fragment of the sequences shown in SEQ ID NOS: 16, 17, 18, and 31. Generally, hybridization conditions are classified into categories, for example very high stringency, high stringency, and low stringency. The conditions corresponding to these categories for probes of approximately 600 bp are provided below.

Very High Stringency
(detects sequences that share 90% sequence identity)

| | | | | | | |
|---|---|---|---|---|---|---|
| Hybridization | in | 5x | SSC | at | 65° C. | 16 hours |
| Wash twice | in | 2x | SSC | at | room | 15 minutes each |
| | | | | | temp. | |
| Wash twice | in | 0.2x | SSC | at | 65° C. | 20 minutes each |

High Stringency
(detects sequences that share at least 80% sequence identity)

| | | | | | | |
|---|---|---|---|---|---|---|
| Hybridization | in | 3x | SSC | at | 65° C. | 16 hours |
| Wash twice | in | 2x | SSC | at | room | 15 minutes each |
| | | | | | temp. | |
| Wash twice | in | 0.5x | SSC | at | 55° C. | 20 minutes each |

Low Stringency
(detects sequences that share at least 50% sequence identity)

| | | | | | | |
|---|---|---|---|---|---|---|
| Hybridization | in | 3x | SSC | at | 65° C. | 16 hours |
| Wash twice | in | 2x | SSC | at | room | 20 minutes each |
| | | | | | temp. | |

Variant PmBiP promoter sequences can be produced by standard DNA-mutagenesis techniques, for example, M13 primer mutagenesis. Details of these techniques are provided in Sambrook et al. (eds.), Molecular Cloning: A Laboratory Manual, 2nd ed., vol. 1-3, Ch. 15, Cold Spring Harbor Laboratory Press, Cold Spring Harbor, N.Y., 1989; and Ausubel et al. (eds.) Current Protocols in Molecular Biology, Greene Publishing and Wiley-Interscience, New York (with periodic updates), 1987. By the use of such techniques, variants can be created that differ slightly from the PmBiP promoter sequences specifically disclosed, yet that still encode a promoter having promoter activity. DNA molecules and nucleotide sequences that are derivatives of those specifically disclosed herein and that differ from those disclosed by the deletion, addition, or substitution of nucleotides while still maintaining promoter activity and/or native PmBiP promoter activity are comprehended by this disclosure.

Transformation

The DNA constructs of the disclosure, containing the PmBiP promoter (SEQ ID NO: 31) or fragments thereof (such as SEQ ID NOS: 16, 17 and 18) operably linked to one or more transgenes may be either homologous or heterologous to the host in question. If homologous to the host cell, i.e., if the transgene is produced by the host cell in nature, then the construct may be connected operably to a different secretory signal sequence and/or terminator sequence than in the natural environment. In this context, the term “homologous” is intended to include a cDNA sequence encoding a transgene that is native to the host cell. The term “heterologous” is intended to include a transgene not expressed by the host cell in nature. Thus, the DNA sequence may be from another organism, or it may be a synthetic sequence.

The host cell of the disclosure, into which the DNA construct or the recombinant expression vector of the disclosure is to be introduced, is any cell capable of driving expression of the PmBiP promoter. Examples of such cells include, but are not limited to, bacteria cells, yeast cells, fungal cells, insect cells, plant cells, and other higher eukaryotic cells.

Various methods of introducing the DNA construct into host cells are well known in the art. For example, in some species, the Ti plasmid of A. tumefaciens can be used to transform host cells (Gouka et al., Nature Biotech. 6:598-602, 1999). The host cell also can be transformed using gene blasting techniques (described above) and standard chemical treatments.

Having illustrated and described the principles of the disclosure in multiple embodiments and examples, it should be apparent to those skilled in the art that the disclosure can be modified in arrangement and detail without departing from such principles. Therefore, the invention includes all modifications coming within the spirit and scope of the following claims.

We claim:

1. An isolated promoter having promoter activity comprising at least 8 promoter elements, wherein the promoter elements are selected from one or more of the group consisting of: E-box motifs (SEQ ID NO: 1), ACGT-core elements (SEQ ID NO: 4), CAAT-boxes (SEQ ID NO: 9), CANABNNAPA elements (SEQ ID NO: 12), HEXMOTIF elements (SEQ ID NO: 27), MNF1 elements (SEQ ID NO: 28), POLLEN1LELAT52 elements (SEQ ID NO: 29), ROOTMOTIF elements (SEQ ID NO: 30), 2SSEEDPROTBANAP elements (SEQ ID NO: 32), BOXIIPCCHS elements (SEQ ID NO: 33), ASF1MOTIF elements (SEQ ID NO: 34), and UPRE elements (SEQ ID NO: 42), wherein at least one of the at least 8 promoter elements is a UPRE element (SEQ ID NO: 42).

2. The isolated promoter of claim 1, wherein the promoter elements are further selected from the group consisting of LTRE elements (SEQ ID NOS: 38 and 39), NRR elements (SEQ ID NO: 40), and QAR elements (SEQ ID NO: 41).

3. The isolated promoter of claim 1, wherein at least one of the at least 8 promoter elements is a BOXIIPCCHS element (SEQ ID NO: 33).

4. The isolated promoter of claim 1, wherein at least one of the at least 8 promoter elements is an ASF1MOTIF element (SEQ ID NO: 34).

5. The isolated promoter of claim 2, wherein at least one of the at least 8 promoter elements is a QAR element (SEQ ID NO: 41).

6. The isolated promoter of claim 2, wherein at least one of the at least 8 promoter elements is a NRR element (SEQ ID NO: 40).

7. The isolated promoter of claim 2, wherein at least one of the at least 8 promoter elements is an LTRE element (SEQ ID NOS: 38 and 39).

8. The isolated promoter of claim 1, wherein the promoter comprises at least 10 promoter elements.

9. The isolated promoter of claim 1, wherein the at least 8 promoter elements are one or more of an ACGT-core element (SEQ ID NO: 4), an E-box motif (SEQ ID NO: 1), CAAT-box (SEQ ID NO: 9); 2SSEEDPROTBANAP element (SEQ ID NO: 32); a CANABNNAPA element (SEQ ID NO: 12); a HEXMOTIF element (SEQ ID NO: 27); a UPRE element (SEQ ID NO: 42); an ASF1MOTIF element (SEQ ID NO: 34), a POLLEN1LELAT52 element (SEQ ID NO: 29), and an MNF1 element (SEQ ID NO: 28).

10. The isolated promoter of claim 9, wherein the at least 8 promoter elements comprise promoter elements in the following order: 5′-ACGT-core element (SEQ ID NO: 4), E-box motif (SEQ ID NO: 1), CAAT-box (SEQ ID NO: 9); 2SSEEDPROTBANAP element (SEQ ID NO: 32) or CANABNNAPA element (SEQ ID NO: 12); HEXMOTIF element (SEQ ID NO: 27), CAAT-box (SEQ ID NO: 9); UPRE element (SEQ ID NO: 42); E-box motif (SEQ ID NO: 1), ASF1MOTIF element (SEQ ID NO: 34), POLLEN1LELAT52 element (SEQ ID NO: 29), and MNF1 element (SEQ ID NO: 28)-3′.

11. A vector, comprising the isolated promoter of claim 1.

12. A host cell, comprising the vector of claim 11.

13. A transgenic plant, comprising the host cell of claim 12.

14. A transgene, comprising the isolated promoter of claim 1 operably linked to an ORF.

15. A vector, comprising a transgene of claim 14.

16. A host cell, comprising the vector of claim 15.

17. A transgenic plant, comprising the host cell of claim 16.

18. The transgene of claim 14, wherein the ORF encodes a protein comprising SEQ ID NO: 36.

19. The transgene of claim 14, wherein the ORF encodes a cationic peptide.

20. The host cell of claim 12, wherein the host cell is a plant cell.

21. The isolated promoter of claim 1, wherein the promoter is inducible.

22. The isolated promoter of claim 21, wherein the promoter is inducible at a temperature of less than 20° C.

23. The promoter of claim 1, wherein the promoter is developmentally specific.

24. The promoter of claim 23, wherein the promoter is expressed in actively dividing cells.

25. The isolated promoter of claim 1, wherein the promoter is wound-inducible.

26. A method for expressing at least one protein in a host cell, comprising introducing the vector of claim 14 into a host cell, wherein the host cell produces a protein from the ORF.

27. The method of claim 26, wherein the host cell is a plant host cell.

28. A protein produced according to the method of claim 26.

29. The protein of claim 28, wherein the protein is a cationic peptide.

30. A method for expressing a protein in a plant, the method comprising growing the transgenic plant of claim 17 under conditions in which to the plant produces a protein encoded by the ORF.

31. The method of claim 30, further comprising wounding the plant.

32. The method of claim 30, further comprising growing the plant at a temperature below 20° C.

33. The method of claim 31, further comprising placing the plant part at a temperature below 20° C.


SEQUENCE LISTING (43 sequences)

SEQ ID NO: 1 - DNA, 6 bp, Artificial Sequence
  variation (3)..(4): N = A, C, G, or T
canntg

SEQ ID NO: 2 - DNA, 6 bp, Artificial Sequence
  Description of Artificial Sequence PROMOTER ELEMENT
gcatgc

SEQ ID NO: 3 - DNA, 29 bp, Artificial Sequence
  Description of Artificial Sequence PROMOTER ELEMENT
aaaaattaatatttaatgttaatattaat

SEQ ID NO: 4 - DNA, 4 bp, Artificial Sequence
  Description of Artificial Sequence PROMOTER ELEMENT
acgt

SEQ ID NO: 5 - DNA, 9 bp, Artificial Sequence
  variation (3)..(4): N = A, C, G, and T
ttnntcatc

SEQ ID NO: 6 - DNA, 13 bp, Artificial Sequence
  Description of Artificial Sequence PROMOTER ELEMENT
aagattcctctaa

SEQ ID NO: 7 - DNA, 10 bp, Artificial Sequence
  Description of Artificial Sequence PROMOTER ELEMENT
gttgttgaga

SEQ ID NO: 8 - DNA, 4 bp, Artificial Sequence
  Description of Artificial Sequence PROMOTER ELEMENT
tata

SEQ ID NO: 9 - DNA, 4 bp, Artificial Sequence
  Description of Artificial Sequence PROMOTER ELEMENT
caat

SEQ ID NO: 10 - DNA, 6 bp, Artificial Sequence
  variation (3): W = a, t, or u
ccwacc

SEQ ID NO: 11 - DNA, 6 bp, Artificial Sequence
  variation (2): R = g or a
grwaaw

SEQ ID NO: 12 - DNA, 7 bp, Artificial Sequence
  variation (2): n=a, t, c, g, or u
cnaacac

SEQ ID NO: 13 - DNA, 11 bp, Artificial Sequence
  variation (1)..(11): w = a, t, or u
wtttatrtttw

SEQ ID NO: 14 - DNA, 9 bp, Artificial Sequence
  Description of Artificial Sequence PROMOTER ELEMENT
ttcgtcatc

SEQ ID NO: 15 - DNA, 9 bp, Artificial Sequence
  Description of Artificial Sequence PROMOTER ELEMENT
tttatcatc

SEQ ID NO: 16 - DNA, 2263 bp, Pseudotsuga menziesii
tcaactaagtccttaatgtcccgagcttttttcttcaccgaccaagatataaacacatcttttaaactcttaaggttcacaacaaactgagatgcaggagatacctctgaactggcatcataaactttccaggatgcttttaataatgctaccaggtcatcatcagctaaccaacaagcattaaacttgaagggactacaagacctgatatcattattcagaatttgtaaataaacaagctgactcgattagaataccctccacacccaacccactgtctaaaataaagatcacaatccaagagatcggccgaaataagtaaccgatccaatcttttacttatgttcttagacctaactctttgattagaccatgttgggagagtaacaacatgaacaatatccactagaccaaatccttccaactgtttaatgaagaaatcagttaatacatctactctagctttgactccccaaacttcggagtatctcagggagaaattcagatccccaccaaaaatcaatttagggcacctaaaacattccaatttcagcagattattccagaataattccctatccaaacaaggaccgtacagattcacaaaacataaatccatctgtagctcaatagaataaagtacaatacacaagtcagaacacaaagcccatgcattgagcaaatgaaatttaagagtcctccaccccaaaaataaacccccagatctacccttggcatccactgacacaaactttcaattctttaacattgtctccaactctcccaccaaaataaccccatcacacatcgtttcttgcaagaaaattacatctaaaaattgctcatcaatcaacctacgcacaactagttttttaggtatactagccaaacccctatagttgagggtcatcaatcataatggaacctctgagggggcaaacactgcgctaagtgccccagtaacagtcttcacttaagaaagcatagttggcataagataatcactaccctgaacaaaaggatgggtccataaagctctcgttctgtaccacacacacgttcttcgttaaccttaaaacaagaccgaaggaaataaccctcactgactacaggattgcctttgtgcagagatacaaaccctagatcccccaatgcaaaatcagacagagccaaattgcaagatgaaagattacccacaacaaaaccctcgatgttcttatccaacaccggttctccaaactcagataacgaggaaatacataagggtttaagagacttcatatagatagactgagaccctaaattcacctcatcaattaatggattaggagaaaggttcgtatccaccaacccatcaatcttgacaataaccggcttatccccagatgtatgccctccattcttaacacgccaaacagatttggtcttatagacagaaccagagttaggttttttcctaaaaggtagagaacaatcatggatcaaatggccataaacatggcatctattacgccgaaatgggatgcccaaataatccaagggttgactaaactcataattaccccttttaatcattaattcaggaacaaggaagaaagaagttacatatctccaatttatctaatttatgttttttttatatacatgctcttgtaaatgttttaaatctctaaatggtataatacgcatcttctacgcaaatatcattcgatttattttcctatatgttttcttacatggcatcaagtccacgtgtagtattgccatttagttaatagatcacacacgtgtccaagtgcaattggttcgaacacctcaagttttcaataataatggacgagcaggaaatgtgggtaattcggagtggttggtcgagaccttccccagtatcttatcaccatgaactaatatttcgaggcggtgacctaaaacaaagaaaataaattaaaagacccattcaattttacccaccgcttttcctacgaggcactaggactacagggaactctcgtaacacgtgtcaataagcgattggctcaaacacgtcaattttttaaaatagctctcaactccgaacgggtaacgtggcgaaatatgagtggaagtactcgacacgtgttggaaagcgatgcgttcagtgacgcatagtgaatttacgggaaagtagatgattctggaagaggtttctaggagcagagtaataagattgtagaagggcaccataaatccattgctctgtgacaaatccttcaaatttggacgc

SEQ ID NO: 17 - DNA, 1259 bp, Pseudotsuga menziesii
taccctgaacaaaaggatgggtccataaagctctcgttctgtaccacacacacgttcttcgttaaccttaaaacaagaccgaaggaaataaccctcactgactacaggattgcctttgtgcagagatacaaaccctagatcccccaatgcaaaatcagacagagccaaattgcaagatgaaagattacccacaacaaaaccctcgatgttcttatccaacaccggttctccaaactcagataacgaggaaatacataagggtttaagagacttcatatagatagactgagaccctaaattcacctcatcaattaatggattaggagaaaggttcgtatccaccaacccatcaatcttgacaataaccggcttatccccagatgtatgccctccattcttaacacgccaaacagatttggtcttatagacagaaccagagttaggttttttcctaaaaggtagagaacaatcatggatcaaatggccataaacatggcatctattacgccgaaatgggatgcccaaataatccaagggttgactaaactcataattaccccttttaatcattaattcaggaacaaggaagaaagaagttacatatctccaatttatctaatttatgttttttttatatacatgctcttgtaaatgttttaaatctctaaatggtataatacgcatcttctacgcaaatatcattcgatttattttcctatatgttttcttacatggcatcaagtccacgtgtagtattgccatttagttaatagatcacacacgtgtccaagtgcaattggttcgaacacctcaagttttcaataataatggacgagcaggaaatgtgggtaattcggagtggttggtcgagaccttccccagtatcttatcaccatgaactaatatttcgaggcggtgacctaaaacaaagaaaataaattaaaagacccattcaattttacccaccgcttttcctacgaggcactaggactacagggaactctcgtaacacgtgtcaataagcgattggctcaaacacgtcaattttttaaaatagctctcaactccgaacgggtaacgtggcgaaatatgagtggaagtactcgacacgtgttggaaagcgatgcgttcagtgacgcatagtgaatttacgggaaagtagatgattctggaagaggtttctaggagcagagtaataagattgtagaagggcaccataaatccattgctctgtgacaaatccttcaaatttggacgcg

SEQ ID NO: 18 - DNA, 263 bp, Pseudotsuga menziesii
ggaactctcgtaacacgtgtcaataagcgattggctcaaacacgtcaattttttaaaatagctctcaactccgaacgggtaacgtggcgaaatatgagtggaagtactcgacacgtgttggaaagcgatgcgttcagtgacgcatagtgaatttacgggaaagtagatgattctggaagaggtttctaggagcagagtaataagattgtagaagggcaccataaatccattgctctgtgacaaatccttcaaatttggacgcg

SEQ ID NO: 19 - DNA, 20 bp, Artificial Sequence
  Description of Artificial Sequence PCR PRIMERS
aatgaaagcgaagtgacacc

SEQ ID NO: 20 - DNA, 24 bp, Artificial Sequence
  Description of Artificial Sequence PCR PRIMERS
cagaaccattaacaagagcaagat

SEQ ID NO: 21 - DNA, 20 bp, Artificial Sequence
  Description of Artificial Sequence PCR PRIMERS
aaccagcagtgataaacgcc

SEQ ID NO: 22 - DNA, 22 bp, Artificial Sequence
  Description of Artificial Sequence PCR PRIMERS
tatggtttggataaaaagggag

SEQ ID NO: 23 - DNA, 30 bp, Artificial Sequence
  Description of Artificial Sequence PCR PRIMER
tcgaagcgcaaatctagagtttaaacttcc

SEQ ID NO: 24 - DNA, 22 bp, Artificial Sequence
  Description of Artificial Sequence PCR PRIMERS
aagaaggcaagctttcaactaa

SEQ ID NO: 25 - DNA, 22 bp, Artificial Sequence
  Description of Artificial Sequence PCR PRIMERS
gcataagaaagcttctaccctg

SEQ ID NO: 26 - DNA, 22 bp, Artificial Sequence
  Description of Artificial Sequence PCR PRIMER
gcactaggaagcttgggaactc

SEQ ID NO: 27 - DNA, 6 bp, Artificial Sequence
  Description of Artificial Sequence PROMOTER ELEMENT
acgtca

SEQ ID NO: 28 - DNA, 8 bp, Artificial Sequence
  Description of Artificial Sequence PROMOTER ELEMENT
gtgccctt

SEQ ID NO: 29 - DNA, 5 bp, Artificial Sequence
  Description of Artificial Sequence PROMOTER ELEMENT
agaaa

SEQ ID NO: 30 - DNA, 5 bp, Artificial Sequence
  Description of Artificial Sequence PROMOTER ELEMENT
atatt

SEQ ID NO: 31 - DNA, 2277 bp, Pseudotsuga menziesii
tctagatcaactaagtccttaatgtcccgagcttttttcttcaccgaccaagatataaacacatcttttaaactcttaaggttcacaacaaactgagatgcaggagatacctctgaactggcatcataaactttccaggatgcttttaataatgctaccaggtcatcatcagctaaccaacaagcattaaacttgaagggactacaagacctgatatcattattcagaatttgtaaataaacaagctgactcgattagaataccctccacacccaacccactgtctaaaataaagatcacaatccaagagatcggccgaaataagtaaccgatccaatcttttacttatgttcttagacctaactctttgattagaccatgttgggagagtaacaacatgaacaatatccactagaccaaatccttccaactgtttaatgaagaaatcagttaatacatctactctagctttgactccccaaacttcggagtatctcagggagaaattcagatccccaccaaaaatcaatttagggcacctaaaacattccaatttcagcagattattccagaataattccctatccaaacaaggaccgtacagattcacaaaacataaatccatctgtagctcaatagaataaagtacaatacacaagtcagaacacaaagcccatgcattgagcaaatgaaatttaagagtcctccaccccaaaaataaacccccagatctacccttggcatccactgacacaaactttcaattctttaacattgtctccaactctcccaccaaaataaccccatcacacatcgtttcttgcaagaaaattacatctaaaaattgctcatcaatcaacctacgcacaactagttttttaggtatactagccaaacccctatagttgagggtcatcaatcataatggaacctctgagggggcaaacactgcgctaagtgccccagtaacagtcttcacttaagaaagcatagttggcataagataatcactaccctgaacaaaaggatgggtccataaagctctcgttctgtaccacacacacgttcttcgttaaccttaaaacaagaccgaaggaaataaccctcactgactacaggattgcctttgtgcagagatacaaaccctagatcccccaatgcaaaatcagacagagccaaattgcaagatgaaagattacccacaacaaaaccctcgatgttcttatccaacaccggttctccaaactcagataacgaggaaatacataagggtttaagagacttcatatagatagactgagaccctaaattcacctcatcaattaatggattaggagaaaggttcgtatccaccaacccatcaatcttgacaataaccggcttatccccagatgtatgccctccattcttaacacgccaaacagatttggtcttatagacagaaccagagttaggttttttcctaaaaggtagagaacaatcatggatcaaatggccataaacatggcatctattacgccgaaatgggatgcccaaataatccaagggttgactaaactcataattaccccttttaatcattaattcaggaacaaggaagaaagaagttacatatctccaatttatctaatttatgttttttttatatacatgctcttgtaaatgttttaaatctctaaatggtataatacgcatcttctacgcaaatatcattcgatttattttcctatatgttttcttacatggcatcaagtccacgtgtagtattgccatttagttaatagatcacacacgtgtccaagtgcaattggttcgaacacctcaagttttcaataataatggacgagcaggaaatgtgggtaattcggagtggttggtcgagaccttccccagtatcttatcaccatgaactaatatttcgaggcggtgacctaaaacaaagaaaataaattaaaagacccattcaattttacccaccgcttttcctacgaggcactaggactacagggaactctcgtaacacgtgtcaataagcgattggctcaaacacgtcaattttttaaaatagctctcaactccgaacgggtaacgtggcgaaatatgagtggaagtactcgacacgtgttggaaagcgatgcgttcagtgacgcatagtgaatttacgggaaagtagatgattctggaagaggtttctaggagcagagtaataagattgtagaagggcaccataaatccattgctctgtgacaaatccttcaaatttggacgcgaaacgcg

SEQ ID NO: 32 - DNA, 7 bp, Artificial Sequence
  Description of Artificial Sequence PROMOTER ELEMENTS
caaacac

SEQ ID NO: 33 - DNA, 7 bp, Artificial Sequence
  Description of Artificial Sequence PROMOTER ELEMENTS
acgtggc

SEQ ID NO: 34 - DNA, 5 bp, Artificial Sequence
  Description of Artificial Sequence PROMOTER ELEMENTS
tgacg

SEQ ID NO: 35 - DNA, 2626 bp, Pseudotsuga menziesii
  CDS (374)..(2338)
gaagctgccgtggtcgcagataatgcaattgcaatgctgaggtttctctgagaggatcgatagtcgggacgattttctctgtttcgatacatatcctttcgcttttcaacgatatcgcttcgttttcagccatttaattcgcatacgtgaacgaagatcggccgcagtgaaggttatcttgtcgatttcgctgttgtgagctttttgcactgcgataacacaccaataggtgtcacttcgctttcattcacgaggtattgaggttgcttctgcttaaaatttgatgcgcgagggttttggaaaggcgccagccatgggacggaagcagaaatgcgctgggttcaacaacgctggaaaagatttcaacggctttatgttccttgcggcgtttatcactgctggttttcttttcagctctgttattgctgcagaagaagcagcaaagttaggaacagtaattggtatagatctcggaaccacgtattcttgtgttggtgtttacaaaaatggtcatgttgaaatcatagcaaatgaccaaggaaataggattacaccttcttgggttgccttcactgataccgaaagactcatcggagaggctgccaaaaaccaggcggcaatgaatcctgaaaggaccgtttttgatgtgaaacggttgattggaagaaagtatgaggacaaggaggtgcaaaaagacatcaaacttttgccctacaaaattgtaaacaaagatgggaagccttacattcaggtgaagatcagggatggtgaaatcaaagtttttagtcccgaggaaattagtgcaatgattttgttgaaaatgaaggaaacagctgagtcctaccttggaaggaaaatcaaggatgcagttgttacagttccagcatatttcaatgatgcacaaagacaggccaccaaggatgctggtgtaattgctgggttaaatgttgctcgtataataaatgagccaactgctgcagcaattgcatatggtttggataaaaagggaggagaaaagaacattcttgtttatgaccttggaggtggaacttttgatgtcagtattctcaccattgataatggtgtttttgaagtgttgtcaaccagcggggatactcatttaggaggagaggacttcgatcaacgagttatggattacttcattaaattggtcaagaaaaaacacaacaaagatattagcaaggataacagagctcttggcaaacttaggagggagtgtgagagggccaaaagagctctgagcagccagcatcaagttcgtgttgaaattgaatcactttttgatggtgttgatttttcagaaccattaacaagagcaagattcgaggaactcaatatggacctcttcaagaaaactcttgggccagtaaagaaggctctagatgatgctaacttgcagaagactgaaattaatgaacttgtgcttgttggaggaagtactcgcataccaaaggttcagcaattattgaaggacttatttgatggcaaggagcctaacaaaggtgttaatccagatgaagctgtggcttatggggctgctgttcagggtggtattctgagtggtgagggaggtgacgaaacaaaagatattcttctattggatgttgctcccctcagcctaggtatagaaactgttggtggagtaatgaccaaacttattccgaggaacactgtcattccaacaaagaagtcacaagtgttcacaacttatcaagatcagcaaaccactgtttcaatcaaggtttatgaaggagagcggagtcttacaaaggattgccgagaattaggcaaatttgatctgtctggaatccctccagctcctcgtggtgtgccacagattgaggtcacctttgaggttgatgccaacggtatcctcaatgtaagagcagaggacaagggcaccaagaaaaccgaaaagattaccatcacaaatgacaaaggtagattgagccaggaagaaatagaaagaatggtcaaggaggcagaggagtttgcagaggaggataagaaagtgaaggacaaaattgatgcgaggaacaatcttgaaacatatgtctacaacatgaaaagcaccattaatgagaaggataaattggcagataaaattgattccgaagacaaggagaagatcgaaactgctatcaaagaagcattggaatggcttgatgacaaccagtcggctgagaaggaggacttcgaggagaagttgaaagaggtggaagctgtatgcagtcccatcatcaagcaagtatatgagaaaactggaggaggatcttctggaggcgatgatgaagacgaggactcgcatgaagaactctaagccatttcagtttctgttgaattttagttgtacaaatcacgatgaactaattctacagaagagatctctgagcataatagggtttatgaggatgattggcaacgaacaagagattcaactgatgaaagtcaaatgactgtttgttttttctatcaatcagaatgttattttcacagattgaaattggcaacgaacaagagattcaactgatgaaagtcaaatgactatttgtttgttttttctatcaatcagaatgttattttcacagatttttcaatctgtagt

SEQ ID NO: 36 - protein, 655 aa, Pseudotsuga menziesii
MFLAAFITAGFLFSSVIAAEEAAKLGTVIGIDLGTTYSCVGVYKNGHVEIIANDQGNRITPSWVAFTDTERLIGEAAKNQAAMNPERTVFDVKRLIGRKYEDKEVQKDIKLLPYKIVNKDGKPYIQVKIRDGEIKVFSPEEISAMILLKMKETAESYLGRKIKDAVVTVPAYFNDAQRQATKDAGVIAGLNVARIINEPTAAAIAYGLDKKGGEKNILVYDLGGGTFDVSILTIDNGVFEVLSTSGDTHLGGEDFDQRVMDYFIKLVKKKHNKDISKDNRALGKLRRECERAKRALSSQHQVRVEIESLFDGVDFSEPLTRARFEELNMDLFKKTLGPVKKALDDANLQKTEINELVLVGGSTRIPKVQQLLKDLFDGKEPNKGVNPDEAVAYGAAVQGGILSGEGGDETKDILLLDVAPLSLGIETVGGVMTKLIPRNTVIPTKKSQVFTTYQDQQTTVSIKVYEGERSLTKDCRELGKFDLSGIPPAPRGVPQIEVTFEVDANGILNVRAEDKGTKKTEKITITNDKGRLSQEEIERMVKEAEEFAEEDKKVKDKIDARNNLETYVYNMKSTINEKDKLADKIDSEDKEKIETAIKEALEWLDDNQSAEKEDFEEKLKEVEAVCSPIIKQVYEKTGGGSSGGDDEDEDSHEEL

SEQ ID NO: 37 - protein, 4 aa, Pseudotsuga menziesii
HEEL

SEQ ID NO: 38 - DNA, 6 bp, Artificial Sequence
  Description of Artificial Sequence PROMOTER ELEMENTS
ccgaaa

SEQ ID NO: 39 - DNA, 5 bp, Artificial Sequence
  Description of Artificial Sequence PROMOTER ELEMENTS
ccgac

SEQ ID NO: 40 - DNA, 8 bp, Artificial Sequence
  Description of Artificial Sequence PROMOTER ELEMENTS
tagtggat

SEQ ID NO: 41 - DNA, 7 bp, Artificial Sequence
  Description of Artificial Sequence PROMOTER ELEMENTS
aacgtgt

SEQ ID NO: 42 - DNA, 21 bp, Artificial Sequence
  Description of Artificial Sequence PROMOTER ELEMENTS
cgaacgggtaacgtggcgaaa

SEQ ID NO: 43 - DNA, 22 bp, Saccaromyces cerevisiae
ggaactggacagcgtgtcgaaa